(19) United States
(12) Patent Application Publication
(10) Pub. No.: US 2018/0072972 A1
(43) Pub. Date: Mar. 15, 2018

(54) SYSTEMS, DEVICES AND METHODS FOR FERMENTING BEVERAGES

(71) Applicant: Alpha Revolution, Inc., San Jose, CA (US)

(72) Inventors: Jong-Wook Shin, San Francisco, CA (US); Yoon-Sang Kim, Seoul (KR); Sang-Hyuck Oh, Seoul (KR); Byung-Kyu Kang, Seoul (KR)

(73) Assignee: Alpha Revolution, Inc., San Jose, CA (US)

(21) Appl. No.: 15/701,241

(22) Filed: Sep. 11, 2017

Related U.S. Application Data
(60) Provisional application No. 62/506,854, filed on May 16, 2017.
(60) Provisional application No. 62/385,663, filed on Sep. 9, 2016.

(51) Int. Cl. C12C 13/10

(52) U.S. Cl. CPC C12C 13/10; C12M 1/36

(57) ABSTRACT

Systems, devices, and methods for the automated production of fermented beverages are provided. The device may be modular, portable and/or self-cleaning. The device includes a housing with chambers that receive a removable fermentation vessel with yeasts and ingredients capable of undergoing fermentation. Flavoring agents and other ingredients may also be added to produce beer, wine, or other alcoholic libations of profound character and freshness. The device includes a controller that initiates fermentation, senses the fermentation status and automatically controls fermentation parameters based on a desired fermentation profile. The controller automatically directs parameters such as carbonation, conditioning, aging, and chilling according to a programmed recipe or consumer preference to create an enjoyable customized beverage.

### CROSS-REFERENCES TO RELATED APPLICATIONS

The present application is a non-provisional and claims the benefit and priority to under 35 U.S.C. § 119(e) to Provisional Application No. 62/506,854, filed May 16, 2017, entitled “Systems, Devices and Methods for Fermenting Beverages,” and U.S. Provisional Application No. 62/385,663, filed Sep. 9, 2016, entitled “Systems and Methods for Fermenting Beverages,” the entire contents of which are incorporated herein by reference for all purposes.

### BACKGROUND OF THE INVENTION

Fermented beverages have been popular drinks throughout history. However, the quality of these libations varies widely between the most vile concoction and the most heavenly nectar. Environmental monitoring and control protocols play an important role in the fermentation process to ensure proper production and consistent quality. Unfortunately, these protocols often require expensive complex equipment, intensive manual labor and/or professional oversight.

Many problems associated with the preparation of alcoholic beverages occur during primary fermentation when yeasts anaerobically convert sugar into carbon dioxide and ethyl alcohol by way of the following chemical reaction:

C₆H₁₂O₆ (sugar) [symbol] 2C₂H₅OH (alcohol)+2CO₂↑ (carbon dioxide)

Secondary fermentation includes, in part, racking (i.e., transferring) the fermenting beverage to another container. This avoids detracting flavors from dying yeast cells, autolysis, and other deleterious processes.

Traditional home brewing of fermented beverages such as beer typically requires a great deal of time, effort, money, and space. It can be messy and often does not result in beers of a desirable quality for consumption. Furthermore, different types of beer require diverse ingredients, fermentation conditions and processes, making it difficult to home brew a suitable variety of type and style of beer.

While devices for home brewing exist, they suffer from many drawbacks. Current home brewing devices tend to oversimplify the important process of primary fermentation and fail to appreciate the nuances of secondary fermentation. Too often, this results in less desirable beer that lack character and freshness.

Information related to attempts to address these problems can be found in U.S. Pat. Nos. 6,032,571; 7,963,213; 8,993,273; 9,109,192; 9,228,163; foreign patent applications: GB 2118571; and GB 2183673; PCT Publication Numbers: WO 2002/014461 A2; WO 2008/020760 A1; WO 2008/143372 A1; and WO 2009/049381 A1 as well as U.S. Patent Application Publication Numbers: US 2004/0129144; US 2015/0000530; US 2015/0000531; US 2015/0000532; and US 2017/0029752, for example. Various systems, devices and methods, including embodiments of the subject invention, can mitigate or solve some or all of these potential problems.

For the foregoing reasons, there's a legitimate need for effective and efficient ways to produce high-quality fermented beverages, including beer, in convenient small batches that would please even the most discriminating connoisseur. Ideally, this would not require professional experience as a brew master or winemaker, expensive, elaborate or delicate equipment, extensive manual labor or inordinate amounts of time. It would be particularly beneficial and desirable to provide a compact, portable, and automated unit that monitors the fermentation process and provides continual and reliable data that is both accurate and precise. Interchangeable containers (e.g., growlers) for dispensing the finished alcoholic beverage (e.g., beer), would also be welcomed. A self-cleaning unit would also be convenient.

### BRIEF SUMMARY OF THE INVENTION

Certain embodiments of the invention relate generally to managing fermentation parameters required to produce alcoholic beverages. More specifically, some embodiments of the present invention provide systems, methods and devices to produce fermented beverages in relatively small batches in an effective and efficient manner to produce high-quality fermented beverages, including beer, in convenient small batches in non-brewery (i.e., home or restaurant) environments. Certain embodiments relate to the versatile creation of types and styles of beer having the correct color, aroma and organoleptic attributes previously experienced only at commercial breweries.

The following presents a simplified summary of some embodiments of the invention in order to provide a basic understanding of the invention. This summary is not an extensive overview of the invention. It is not intended to identify key/critical elements of the invention or to delineate the scope of the invention. Its sole purpose is to present some embodiments of the invention in a simplified form as a prelude to the more detailed description that is presented later.

In one aspect, a fermentation device is provided. The device comprises a fermentation vessel with an open and a closed fermentation configuration and a specific gravity measurement system comprising a weight sensor and a pressure sensor. The specific gravity measurement system configured to sense the level of fermentation in the open or the closed fermentation configuration. A fermentation controller is configured to access a desired brix profile, receive the sensed level of fermentation in the open or the closed fermentation configuration from the specific gravity measurement system, and adjust a fermentation parameter during a primary and/or a secondary fermentation of a mixture contained in the fermentation vessel in real time. The fermentation parameter comprises at least one of a temperature of the fermentation vessel, a pressure of the fermentation vessel, a specific gravity of the mixture, a brix of the fermentation mixture, and the fermentation configuration. The fermentation controller is further configured to receive a weight sensed by the weight sensor of the specific gravity measurement system before primary fermentation begins, receive the specific gravity of the fermentation mixture calculated from a beginning volume of the mixture in the fermentation vessel and a starting specific gravity obtained from a tag, convert the weight drop sensed from the weight sensor over a period of time to specific gravity during the primary fermentation using a first formula, determine a first fermentation status of the mixture in the fermentation vessel based on the converted specific gravity, and adjust the fermentation parameter during the primary fermentation of the mixture in the fermentation vessel based on the first fermentation status and a desired fermentation status.

In additional aspects, the fermentation controller is further configured to receive a pressure of carbon dioxide sensed by the pressure sensor of the specific gravity measurement system when the fermentation vessel is in the closed fermentation configuration at the secondary fermentation, receive a volume of carbon dioxide from the fermentation device, convert the pressure and the volume of carbon dioxide to specific gravity using a second formula, convert the volume of carbon dioxide to an amount of brix using a third formula, determine a second fermentation status of the mixture in the fermentation vessel based on brix and/or specific gravity, and adjust the fermentation parameter during the secondary fermentation of the mixture in the fermentation vessel based on the current fermentation status and a desired fermentation status. The first formula is specific gravity=weight/volume, the second formula is sugar weight change=(from 90 n to 95.74 n), PV=nRT→n=PV/RT, and the third formula is specific gravity change=sugar weight change/initial wort volume. The fermentation controller is configured to determine the first or the second fermentation status of the mixture based on a mean value of the specific gravity during a predetermined time interval. The fermentation controller is configured to determine the first or the second fermentation status of the mixture based on a change in the specific gravity during a predetermined interval. The first or the second fermentation status of the mixture is determined in real time. The tag is a QR code, a barcode, an RFID tag, an NFC tag or any other near field data communication mechanism affixed to an outside surface of the fermentation vessel. The desired fermentation status is determined based on the accessed desired brix profile, wherein the accessed desired brix profile comprises a desired brix profile for a particular type of beer. The first or the second fermentation status comprises at least one of an amount of sugar consumption in the fermentation vessel and a fermentation rate of the mixture in the fermentation vessel. The device is a portable and a self-contained fermentation device.

In yet another aspect, a portable beer fermentation device is provided. The device comprises a housing with a main chamber for removably receiving a fermentation growler and an ingredients chamber for removably receiving at least one pod, the ingredients chamber in fluid connection with the fermentation growler. A pressurized fluid source is in fluid communication with the ingredients chamber and the fermentation growler. The pressurized fluid comprises filtered air and the fermentation growler is removably received in the main chamber. The fermentation growler contains a mixture capable of undergoing fermentation. A control module is configured to actuate the pressurized fluid source to transfer contents of the at least one pod into the fermentation growler, receive an specific gravity measurement of the mixture, and control at least one of a temperature and a pressure of the fermentation growler based on the specific gravity measurement so as to cause the mixture to undergo a fermentation to yield an enjoyable, characteristic beer. The at least one pod includes a yeast pod, a hop pod, a flavoring pod, a color pod, and/or a pod with any other ingredients. The mixture capable of undergoing fermentation includes a malt mixture, liquid malt extract, dry malt extract or wort.

The portable beer fermentation device also comprises a dispenser in fluid connection with the fermentation growler. The dispenser is configured to dispense the yielded beer to a desired location outside of the housing. The ingredients chamber is configured to keep the at least one pod immersed in the mixture contained in the fermentation growler for a predetermined time so as to alter the taste, flavor, aroma, color or other characteristics of the beer. The device is configured to move the mixture between the fermentation growler and the ingredients chamber multiple times such that the flavor and/or the color of the beer is increased each time the mixture is moved. The fermentation growler of the portable beer fermentation device is configured to be disposed within the housing or disposed outside the housing, wherein a mobile dispenser allows beer to be served from the fermentation growler when the growler is disposed outside the housing and a dispensing module allows beer to be served from the fermentation growler when the growler is disposed inside the housing and connected to the portable beer fermentation device.

The portable beer fermentation device also includes a locking cap configured to seal a fermentation growler and a check valve assembly disposed within the locking cap, the check valve assembly configured to open a beer flow path and a gas flow path of the fermentation device when the locking cap engageably seals the fermentation growler and to close the beer flow path and the gas flow path of the fermentation device when the locking cap disengageably unseals the fermentation growler such that inadvertent release of the beer and/or unintentional entry of contaminants are prevented. The beer can include any combination of a type and a style of the beer as chosen by a consumer. The beer type is selected from the group consisting of an ale and a lager; and the beer style is selected from the group consisting of bock, doppelbock, pilsner, rauchbier, porter, stout, iambic, amber, blonde, light, white, pale, red, golden, brown, dark, saison, cream, fruit, bitter, honey, IPA, lime, and strong, for example.

The portable beer fermentation device further comprises one or more processors and a non-transitory computer-readable medium containing instructions that, when executed by the one or more processors, cause the one or more processors to perform operations. The operations performed includes receiving an input that controls the desired fermentation, identifying a status of the desired fermentation, and notifying a completion of the desired fermentation based on the input. The portable beer fermentation device further comprises instructions that, when executed by the one or more processors, cause the one or more processors to perform operations. The operations including: place orders for supplies; access information about fermented beverages; access food pairing information; purchase accessories; make recommendations; connect with social media; and provide feedback. The instructions are modified by the one or more processors based on feedback received from at least one sensor. The at least one sensor is configured to monitor parameters and is selected from the group consisting of a temperature sensor, a weight sensor, a pressure sensor, an identification sensor, an air quality sensor, an SG sensor, and a level sensor. The parameters are selected from the group consisting of temperature, weight, pressure, orientation, yeast, gas, and water.

In yet another aspect, a method for making a fermented beverage using a portable fermentation device is provided. The method comprises providing a portable fermentation device, accessing a recipe, placing primary ingredients into a fermentation vessel, the vessel removably positioned inside the portable fermentation device, adjusting at least one fermentation parameter, opening the fermentation vessel, fermenting one or more of the primary ingredients, measuring brix and/or specific gravity of the ingredients, determining fermentation status based on the step of measuring brix and/or specific gravity of the ingredients, closing the fermentation vessel at a desired fermentation, optionally adding carbonation, adjusting a serving temperature, and dispensing the fermented beverage from the portable fermentation device so as to be enjoyed by a consumer. The method further comprises optionally placing additional ingredients into a dry hopping module after the step of determining fermentation status based on brix and/or specific gravity measurement and before the step of closing the fermentation vessel and optionally aging after the step of adding carbonation and before the step of adjusting a serving temperature. The method further comprises adding secondary ingredients to a dry hopping system of the portable fermentation device before the step of dispensing the fermented beverage. The secondary ingredients include hops, flavoring agents, coloring agents, clarifying agent, or any combination thereof.

The primary ingredients include yeast and at least one substance capable of undergoing fermentation. The substance(s) capable of undergoing fermentation is a grain-based wort or a fruit and the fermented beverage is a beer or a wine, respectively. The recipe includes accessing a pre-programed recipe or customizing a recipe to fit a personal taste preference of the consumer. The method further comprises replacing the fermentation vessel with a sanitizing cap, replacing the optional additional ingredients with a sanitizing pod, and adding water to dry hopping module to begin cleaning the fermentation device.

These and other features, aspects, and advantages of various embodiments of the invention will become better understood with regard to the following description, appended claims, accompanying drawings and abstract.

### BRIEF DESCRIPTION OF THE DRAWINGS

FIG. 1A shows a perspective view of a portable fermenting device including interactions with remote devices according to embodiments of the invention.

FIG. 1B shows an alternative view of a portable fermenting device according to embodiments of the invention.

FIG. 1C shows insertion of pods into the ingredients chamber of the portable fermenting device shown in FIGS. 1A and 1B according to some embodiments of the invention.

FIG. 1D shows insertion of a fermentation vessel into the portable fermenting device shown in FIGS. 1A and 1B according to some embodiments of the invention.

FIG. 1E shows a schematic representation of the control system, dry hopping module, level sensor, lid, and other components of a portable fermenting device of FIGS. 1A-1D according to embodiments of the invention.

FIG. 2A shows a schematic representation of a portable fermentation device according to certain embodiments of the invention.

FIG. 2B shows a schematic representation of a portable fermentation device according to some embodiments of the invention.

FIG. 2C is a cross-sectional view showing selected internal components of the portable fermentation device according to certain embodiments of the invention.

FIG. 3A shows exemplary brix/specific gravity and fermentation level profiles during primary and secondary fermentation according to embodiments of the invention.

FIG. 3B is a flow diagram according to embodiments of the invention.

FIG. 4A is a bottom view of the device shown in FIGS. 1A and 1B according to embodiments of the invention.

FIG. 4B is a magnified cross sectional view of a load cell according to embodiments of the invention.

FIG. 4C is a bottom view of the device shown in FIG. 4A with the housing and faucet removed to show load cell sensors and their corresponding connections according to some embodiments of the invention.

FIG. 5A shows the interaction of a locking structure between a cap assembly and a closed cover of a portable fermentation device according to embodiments of the invention.

FIG. 5B shows the interaction of a locking structure between a cap assembly and an open cover of a portable fermentation device according to embodiments of the invention.

FIG. 6A shows a locking cap and a check valve assembly sealing a fermentation vessel of a portable fermentation device according to embodiments of the invention.

FIG. 6B shows a locking cap detail of FIG. 6A according to embodiments of the invention.

FIG. 7A is a diagram of a dry hopping system according to embodiments of the invention.

FIG. 7B is a diagram of a dry hopping structure in relation to FIG. 7A according to some embodiments of the invention.

FIGS. 7C and 7D show the interaction of an ingredients pod of the dry hopping structure as shown in FIG. 7B according to some embodiments of the invention.

FIG. 7E is a diagram showing degradation prevention in relation to FIG. 7A according to some embodiments of the invention.

FIG. 8A is a diagram of a cleaning circulation system according to embodiments of the invention.

FIG. 8B is a diagram of a cleaning detail related to FIG. 8A according to embodiments of the invention.

FIG. 8C is a diagram of the cleaning system loop with flow in a clockwise direction according to embodiments of the invention.

FIGS. 8D is a diagram of the cleaning system loop with flow in a counterclockwise direction according to embodiments of the invention.

FIG. 8E is a diagram of the cleaning system loop with flow in a draining direction according to embodiments of the invention.

FIG. 9A is a cross sectional diagram of the dry hopping module including a separable dry hopping tank according to embodiments of the invention.

FIG. 9B shows the relationship between a separable dry hopping tank and top lid according to some embodiments of the invention.

FIG. 9C shows the top lid covering the separable dry hopping tank according to some embodiments of the invention.

FIG. 10A is a cross sectional diagram showing cooling air flow around the fermentation vessel according to embodiments of the invention.

FIG. 10B is a perspective view of the fermentation vessel holder showing air flow around the holder according to embodiments of the invention.

FIG. 10C is a side view of FIG. 10B showing air flow through and around the fermentation vessel holder according to embodiments of the invention.

FIG. 10D is a side view of the fermentation vessel holder according to embodiments of the invention.

FIG. 10E is a top view of FIG. 10D according to embodiments of the invention.

FIG. 11A shows the inside cover of an automatic locking system of the portable fermenting device according to some embodiments of the invention.

FIG. 11B shows a switch of an automatic locking system of the portable fermenting device according to embodiments of the invention.

FIG. 11C is a cross sectional diagram of an automatic locking system of the portable fermenting device with a partially disengaged pin according to embodiments of the invention.

FIG. 11D shows a cross sectional diagram of an automatic locking system of the portable fermenting device with a fully engaged pin according to embodiments of the invention.

FIG. 11E is a magnified view of part of FIG. 11D according to embodiments of the invention.

FIG. 12A is a cross sectional diagram of a manual locking system of the portable fermenting device according to embodiments of the invention.

FIG. 12B is a perspective view of a cap, sanitizer bottle, and sanitizer gripper of the manual locking system of the portable fermenting device according to embodiments of the invention.

FIG. 12C is a cut-a-way perspective view of FIG. 12B according to embodiments of the invention.

FIG. 12D is an exploded view of FIG. 12B according to embodiments of the invention.

FIGS. 13A-13C show the sequential steps to manually attach the cap, sanitizer bottle, and sanitizer gripper of the manual locking system to the inside cover of the portable fermenting device according to embodiments of the invention.

FIG. 13D shows the inside cover of the portable fermenting device in an open position with the cap, sanitizer bottle, and sanitizer gripper attached according to some embodiments of the invention.

FIG. 13E is a cut-a-way perspective view of FIG. 13D according to embodiments of the invention.

FIG. 14A is a perspective view of the fermentation vessel detached from a mobile dispenser according to embodiments of the invention.

FIG. 14B is a perspective view the fermentation vessel partially attached to a mobile dispenser according to embodiments of the invention.

FIG. 14C is a side view the fermentation vessel fully attached to a mobile dispenser according to some embodiments of the invention.

FIG. 14D is a cross sectional diagram of the locking mechanism to attach the fermentation vessel to the mobile dispenser according to embodiments of the invention.

### DETAILED DESCRIPTION OF THE INVENTION

The following description is presented to enable any person skilled in the art to make and use the embodiments, and is provided in the context of a particular application and its requirements. Various modifications to the disclosed embodiments will be readily apparent to those skilled in the art, and the general principles defined herein may be applied to other embodiments and applications without departing from the spirit and scope of the disclosure. Thus, the invention is not limited to the embodiments described and shown.

The process of home brewing beer typically includes three major phases, (1) brewing and processing wort, which is the malt and sugar solution prior to fermentation, (2) primary fermentation of the wort which converts the malt sugars into alcohol and releases CO₂, and (3) conditioning which may include additional fermentation, carbonation, and aging as desired. While much attention has been given to the first phase, the focus of this disclosure is the fermentation and conditioning, which plays a significant role in the quality of the beer produced.

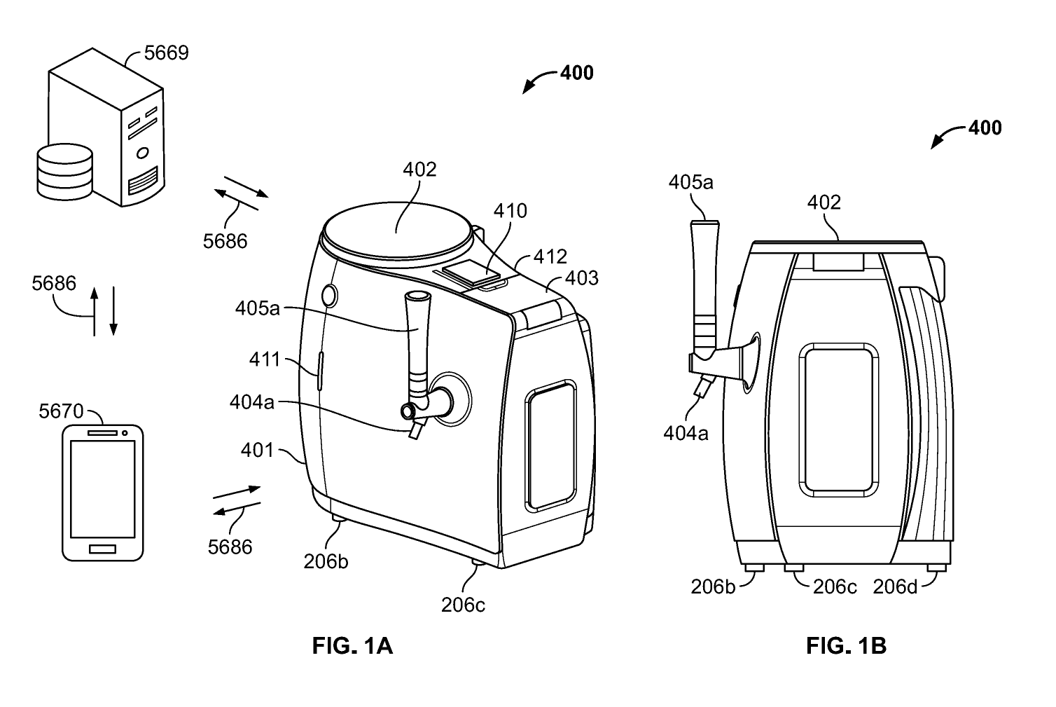

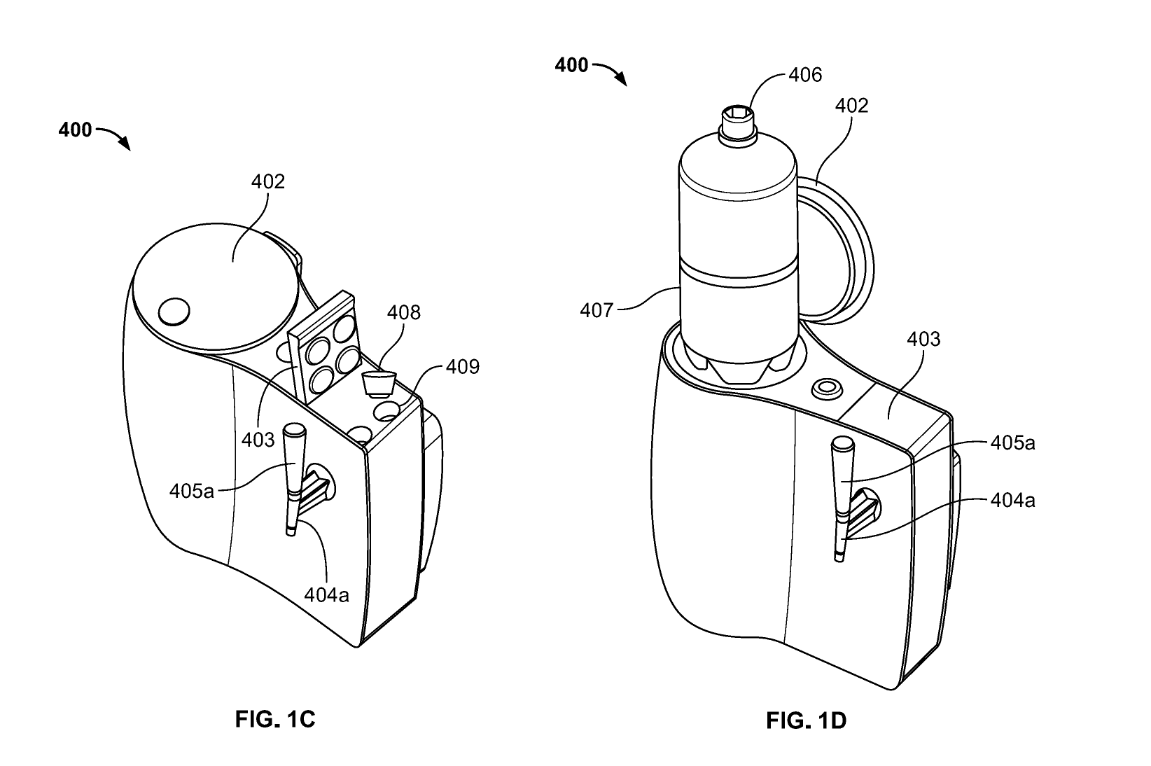

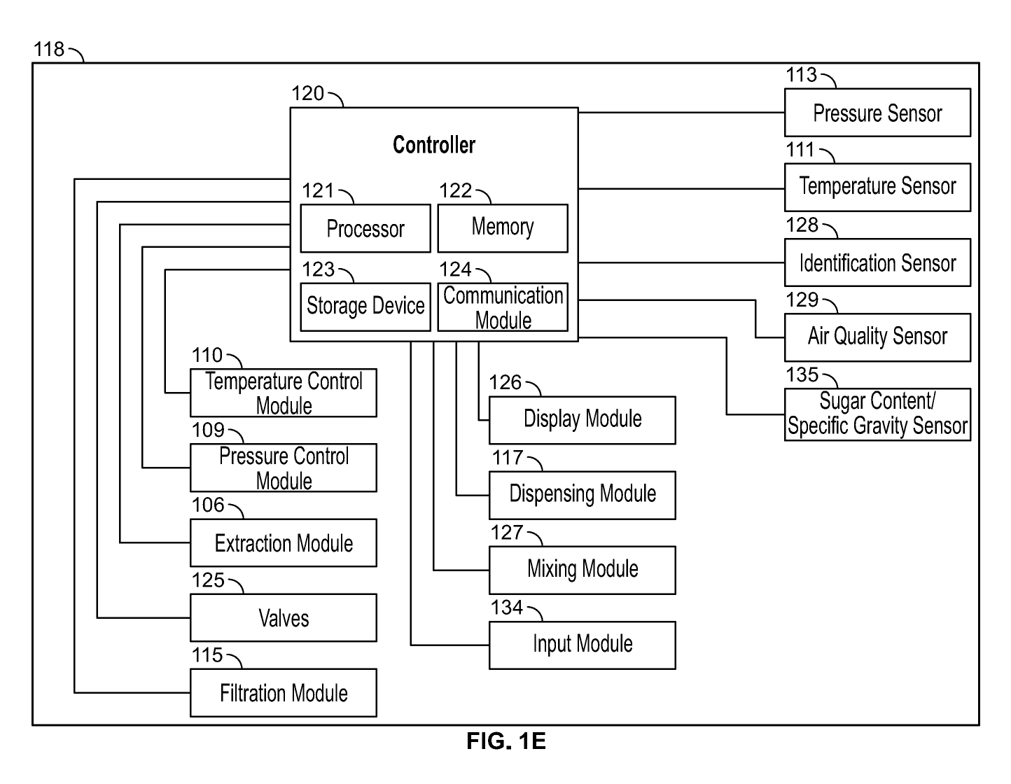

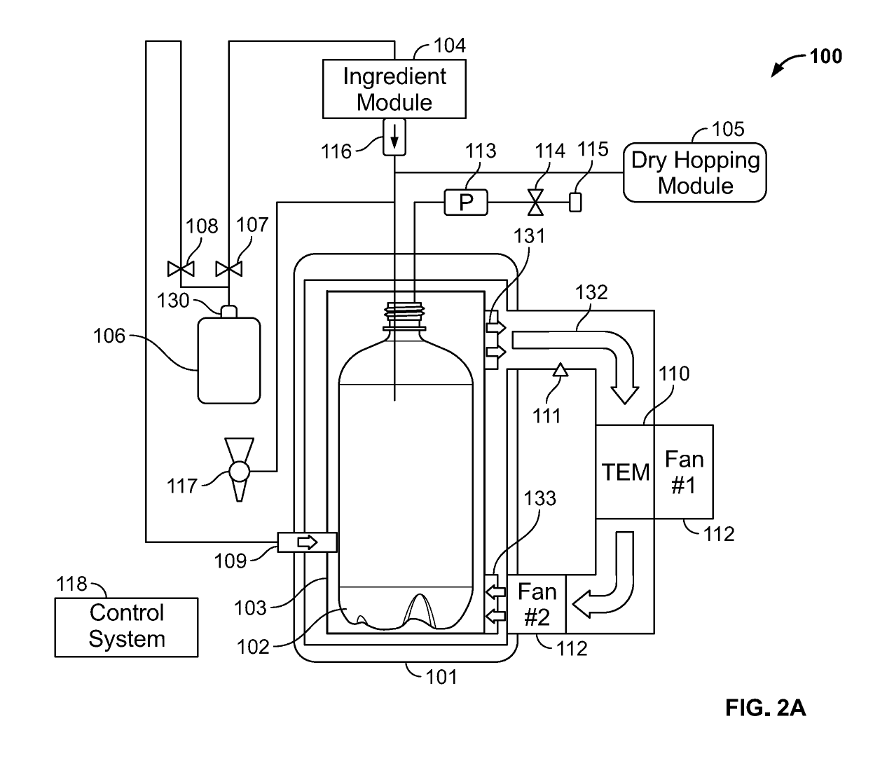

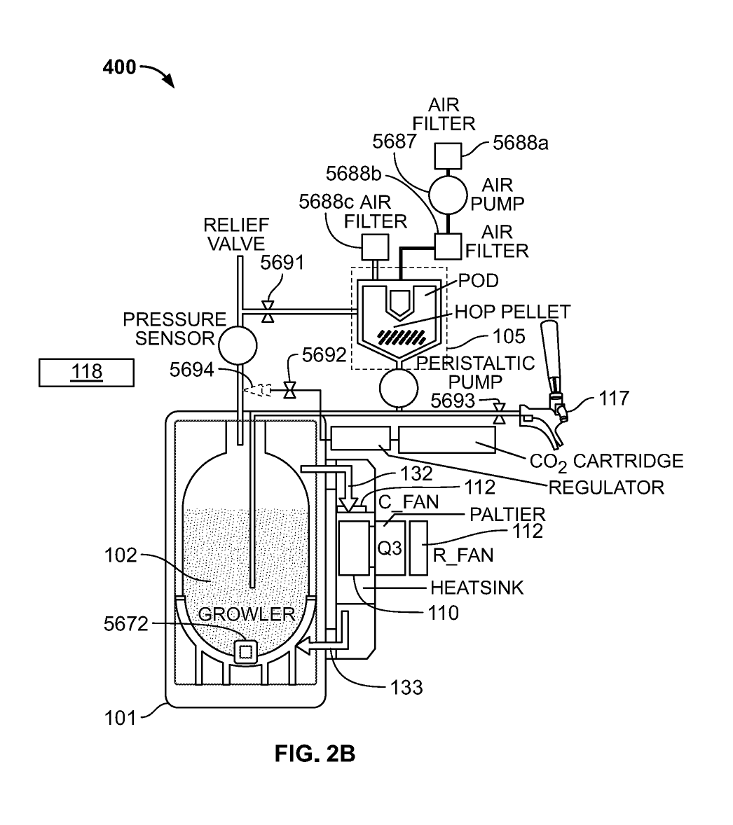

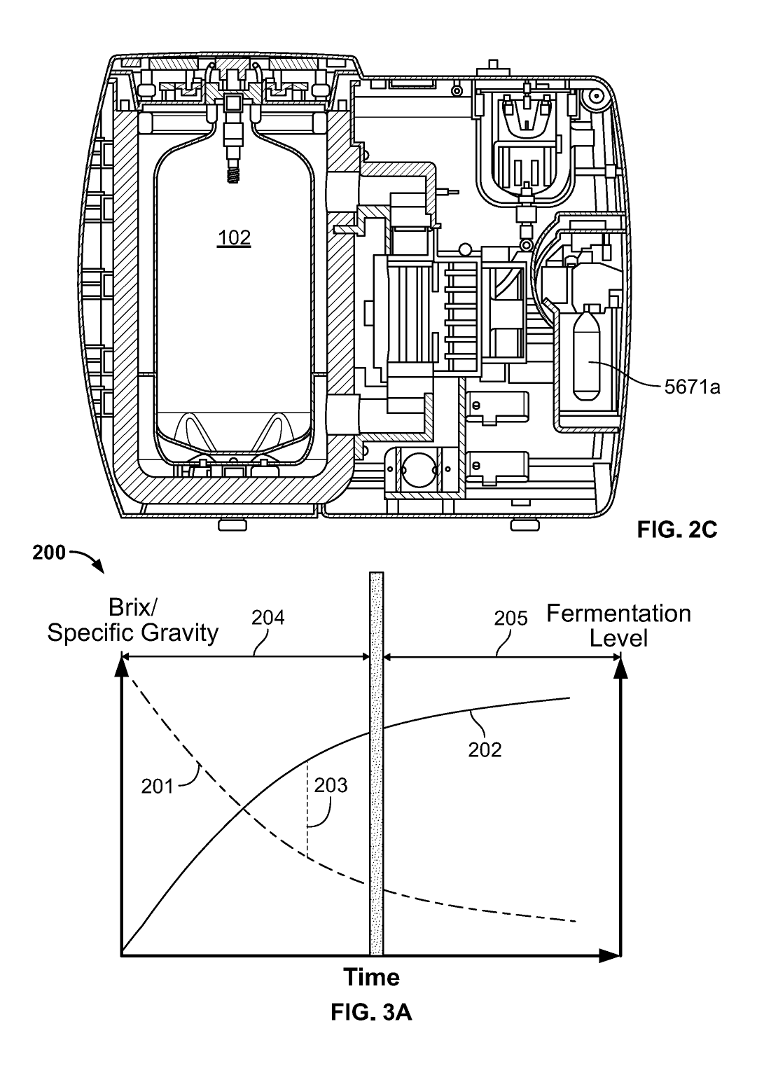

FIGS. 1A-1D show a portable fermenting device 400 according to embodiments of the invention. It is contemplated that this fermenting device may be used to produce any number of alcoholic beverages requiring fermentation, including beer, for example. Device 400 may include generally the same components as device 100 described below and shown in FIG. 2A. It may also operate generally the same as device 100. As can be seen in FIG. 1A, device 400 includes housing 401, faucet 404a, and tap pull 405a for dispensing fermented beverages, including beer, once fermentation is completed. As can be seen with reference to FIGS. 1A and 1D, a cover 402 may provide access to a main chamber 407 which may receive and hold fermentation vessel 406. Similarly, as seen with reference to FIGS. 1A and 1C, a cover 403 may provide access to a ingredients chamber 409 which may receive and hold pods 408 that may contain ingredients such as yeast, hop oil or other forms of hops color, flavors and other ingredients as needed. A display 410 and controls 412 may be used to operate the system and status bar 411 may be used to track the progress of the fermentation. The fermentation system 400 may be remotely linked to peripheral devices including a computer server 5669 or smart phone 5670, for example. The remote link 5686 may be WiFi, Bluetooth®, or similar technology.

FIG. 1E shows a schematic representation of the control system 118 and other components of the portable fermenting device 100, 400 of FIGS. 1A and 1D according to embodiments of the invention. As can be seen in FIG. 2A, fermenting device 100 may include a housing 101 with a fermentation vessel 102 disposed therein. In the case of brewing, fermentation vessel 102 may be a pre-packaged fermentation growler including a malt and sugar solution (e.g., wort) used as a starting point for generating a fermented beverage such as beer. In some embodiments, housing 101 may have an inner housing 103 disposed within housing 101 and surrounding fermentation vessel 102 and the inner housing 103 may be connected to an air pump that can compress any vessel (such as fermentation vessel 102) that is disposed inside inner housing 103. In some embodiments, the starting contents of the fermentation vessel 102 may be wort in its full liquid state. In some embodiments, the starting contents may be a liquid malt extract generated by compressing wort to reduce the volume for distribution purposes. In some embodiments, the starting contents may be a dry malt extract generated by dehydrating wort for distribution processes. In some embodiments, the malt and sugar solution may be specifically selected to generate a particularly desired type of beer, such as an ale or lager or specific style thereof. For example, the malt and sugar solution provided in fermentation vessel 102 may be specific to an ale such as a pale ale, India pale ale, stout, porter, brown ale, wheat, or Belgian, or a lager such as a light lager, dark lager or bock. Any of the various types or styles of beers may also have particular flavoring, hops, or yeast capsules as described below. It will be understood that although referred to primarily as a beer fermenting device for illustrative purposes, other fermented beverages may also be generated by device 100 and any other fermenting devices described herein. For example, device 100 may be configured to yield fermented ciders made from apples or pears or other fermented beverages as will be understood by those skilled in the art. Various embodiments of fermentation vessel will be described further below with reference to the figures therein.

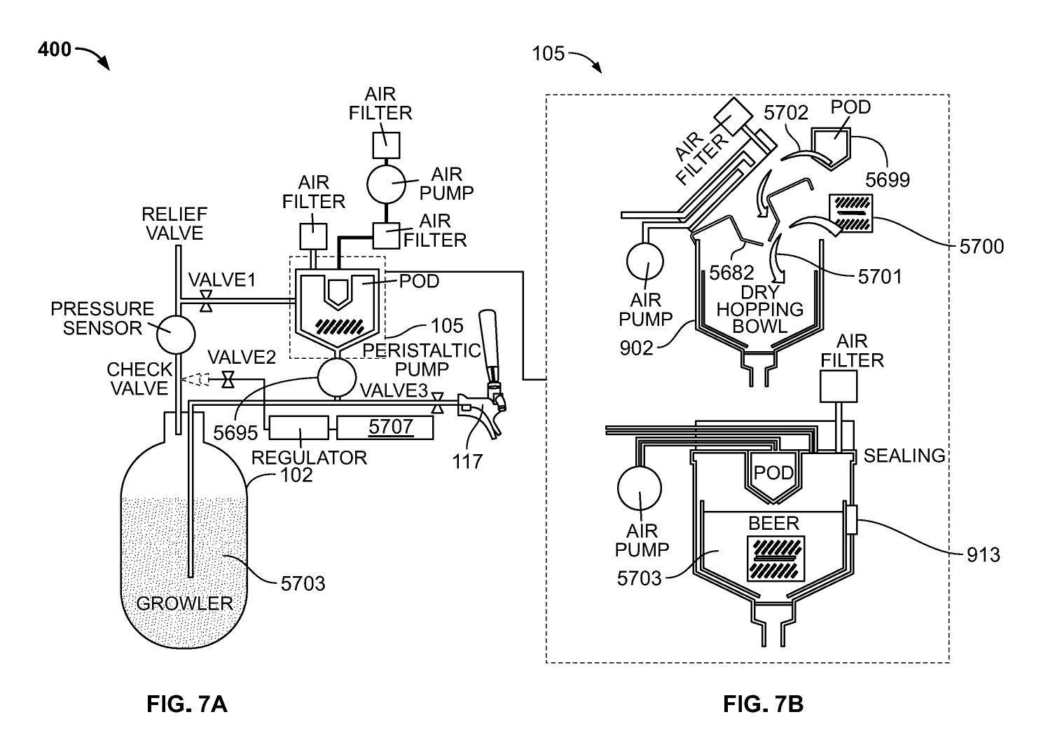

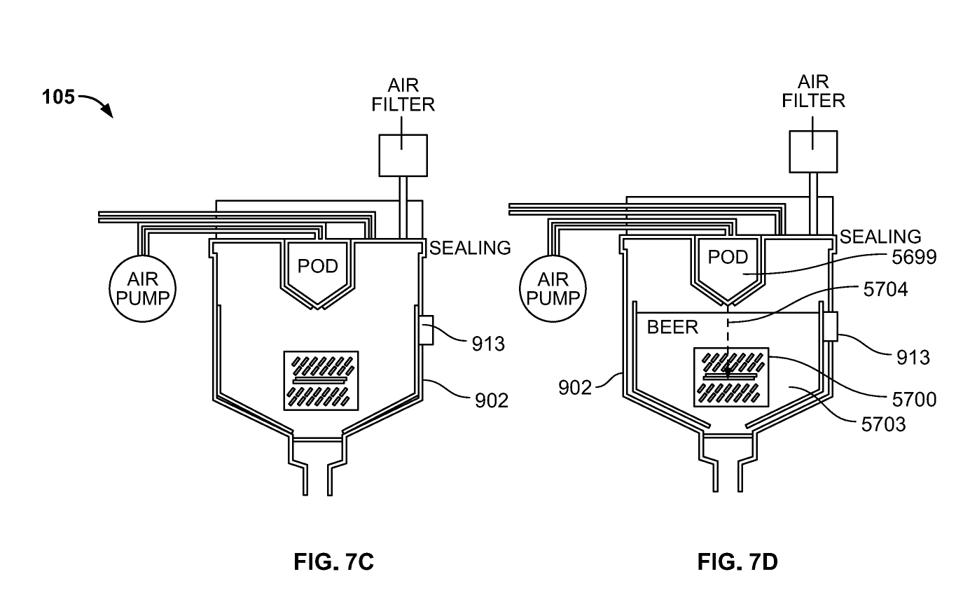

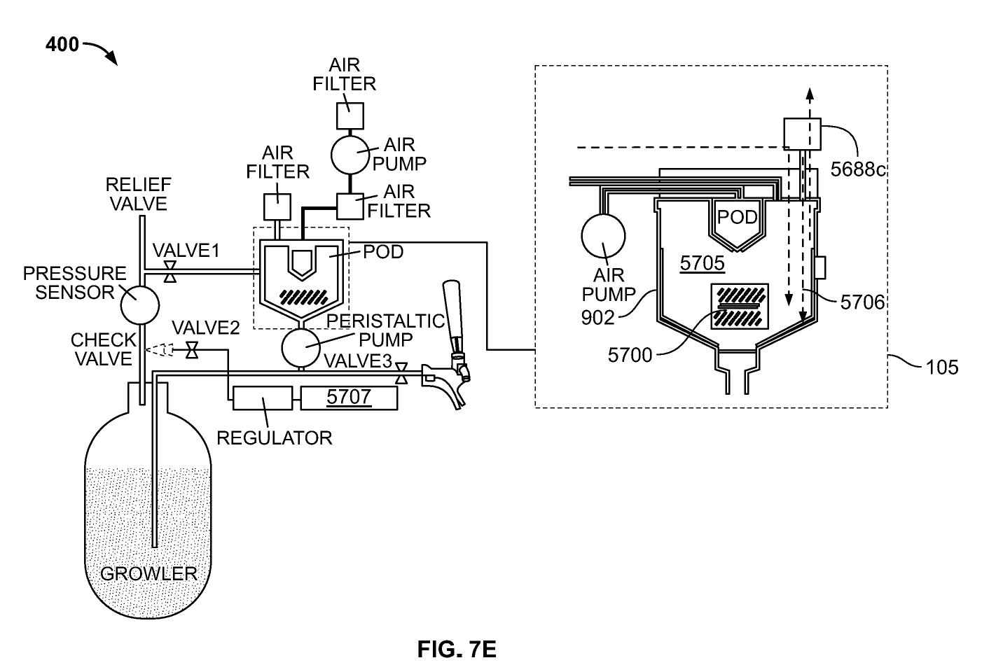

Portable fermenting device 100 may further include (also within housing 101, in some embodiments) ingredients module 104. Ingredients module 104 may utilize pods containing additional ingredients to be mixed with the contents of fermentation vessel 102 to generate a desired beer. For example, ingredients module 104 may utilize pods with ingredients such as yeast, hops or hop oil, flavoring ingredients, coloring ingredients, and/or any other ingredients needed. As with the malt and sugar solution described above, the ingredients module 104 may be specifically selected for a specific type or style of beer desired. As can be seen in FIG. 2A, ingredients module 104 may be in fluid communication with fermentation vessel 102, with check valve 116 disposed in between to control the direction of flow. Specifically, as can be seen in FIG. 2A, check valve 116 ensures that the direction of flow allows contents of ingredients module 104 to travel in the direction of the fermentation vessel 102 and does not allow the contents of fermentation vessel 102 to flow to ingredients module 104. In some embodiments, the contents of ingredients module 104 may be forced into fermentation vessel 102 by actuation of pressurized fluid from pump 106. Specifically, fluid pumped by pump 106 may travel to ingredients module 104 when valve 107 is opened, and cause the contents thereof to flow into fermentation vessel 102. While pressurized fluid may be any suitable liquid or gas, preferably, pump 106 is an air pump that forces pressurized air to release the contents of ingredients module 104. It will be understood that in contrast with pumping liquids such as water, use of an air pump may allow for easier maintenance of device 100 since residue left within the channels may be minimized. As can be seen in FIG. 2A, a filter 130 may be provided between air pump 106 and valves 107 in order to avoid contamination of the ingredients in ingredients module 104, fermentation vessel 102 and/or dry hopping module 105. In addition to or in place of the ingredients module 104, in some embodiments the portable beer fermenting device 100 may also include dry hopping module 105. As will be described in further detail with reference to FIGS. 2B, and 7A-7D, a dry hopping module 105 may further supplement the taste, flavor, aroma, color and/or any other aspects of the beverage by siphoning wort into the module at a desired time in the beer making process.

Pump 106 may also be in fluid communication with a pressure control module 109. In some embodiments, pressure control module 109 may be configured to apply pressure within fermentation vessel 102. For example, as depicted in FIG. 2A, pressure control module 109 may cause pump 106 to provide pressurized fluid (such as air) to inner housing 103 when valve 108 is opened so as to place indirect pressure on the contents of fermentation vessel 102. It will be understood that pressure control module 109 may be any suitable pressure control device configured to apply or relieve pressure from fermentation vessel 102. Depending on the type of fermentation vessel 102 employed by a given device (as described below), pressure control module 109 may be suitably configured to control the pressure therein. Pressure control module 109 may be used to apply indirect pressure on the contents of fermentation vessel 102 so as to controllably force beer out of vessel 102 via dispenser 117 once the beer is completed. It will be understood that the indirect pressure applied by pressure control module 109 via air pump 106 may provide advantages over conventional CO₂ or N₂ systems used to directly pressurize beer. For example, use of indirect pressure maintains the taste of the beer by avoiding any reaction between the air and the beer and the need for replacing the fluid source (such as CO₂ or N₂), while allowing for control of the pressure in fermentation vessel 102 and pressurized fermentation of the contents of fermentation vessel 102 (and ingredients module 104).

Device 100 may also include a temperature control module 110 to control the temperature of fermentation vessel 102. Temperature control module 110 may include cooling or heating elements 112 to provide cooling and heating of fermentation vessel 102 as needed via its surrounding chamber as shown in FIG. 2A. For example, temperature control module 110 may include any suitable refrigeration and/or heating components that transfer heating and/or cooling to and from the chamber surrounding fermentation growler as depicted by arrows 131, 132, and 133. Temperature control module 110 may include temperature sensor 111, which may allow for precise feedback control of the temperature of fermentation vessel 102 during fermentation. Although not shown in FIG. 2A, it will be understood that in some embodiments temperature control module may provide direct heating/cooling via contact with the fermentation vessel 102 and/or indirect heating/cooling via airflow such as the heat sink/fan configuration shown in FIG. 2B, for example. Of course, a plethora of additional or alternative configurations are possible some of which are shown in FIG. 2B, for example.

As can be seen in FIG. 2A, fermentation vessel 102 may be configured to be an open or closed fermentation vessel depending on the position of valve 114. For example, if valve 114 is in the open position, filtered air may flow through vessel 102 via filtration module 115. Filtration module 115 may be any suitable air filter designed to keep out substances that may adversely affect fermentation. It will be understood that controlled open fermentation may have certain advantages. For example, with controlled open fermentation, the device can release undesirable off flavors that may be generated during the fermentation process and prevent contamination of the fermentation vessel, which may otherwise require complicated equipment and/or highly skilled individuals in a commercial brewing setting. On the other hand, if valve 114 is in the closed position, air will not be able to travel into vessel 102 via filtration module 115, and vessel 102 will act as a closed fermentation vessel. As will be described below in further detail, controlled opening and closing of valve 114 may allow for precise control of fermentation and carbonation to provide extremely fresh beer that exhibits authentic characteristics.

Once the contents of fermentation vessel are fermented, carbonated, and chilled to the appropriate temperature, the resulting beer may be directly removed from device 100 for drinking via dispenser 117 (using pressure control module 109 as described above). Dispenser 117 may include any suitable beverage dispenser, including, for example, a faucet with a tap handle as can be seen in FIGS. 1A-1D.

As noted above, traditional home brewing typically requires large containers and equipment that tend to take up excessive space and do not allow for generation of smaller batches of beer. Accordingly, the devices described herein, including device 100 described above has the advantage of being compact and portable. For example, device 100 (and devices described below) may be sized to fit on a typical kitchen countertop and easily moved by a user. For example, device 100 and devices described below may have dimensions that allow the fermentation vessel to hold 3 to 6 L of beer. In some embodiments, device 100 and devices described below may be expandable as described in U.S. Provisional Patent Application Ser. No. 62/385,663, filed Sep. 9, 2016, entitled Systems and Methods for Fermenting Beverages, the disclosures of which are hereby incorporated by reference in their entirety for all purposes.

The various components of device 100 may be controlled by control system 118. Although shown illustratively as separate from components of device 100, control system 118 may be connected to and include components of device 100 as will be described in further detail with reference to FIG. 1E. Specifically, control system 118 can facilitate optimal fermentation, carbonation, conditioning, chilling, storing and serving of beer in accordance with embodiments of the invention. Control system 118 can include a main controller 120 and a plurality of modules that may communicate with one another via a wired or wireless communication channel. Control system 118 (and components thereof) may be realized using one or more integrated circuits and/or printed circuit boards, and may include fewer or more modules than those shown in FIG. 1E. Further, control system 118 may be integrated in a computer system, or realized as a separate device which is capable of communicating with other computer systems (e.g., computer servers 5669) and/or devices (e.g., smart phones 5670).

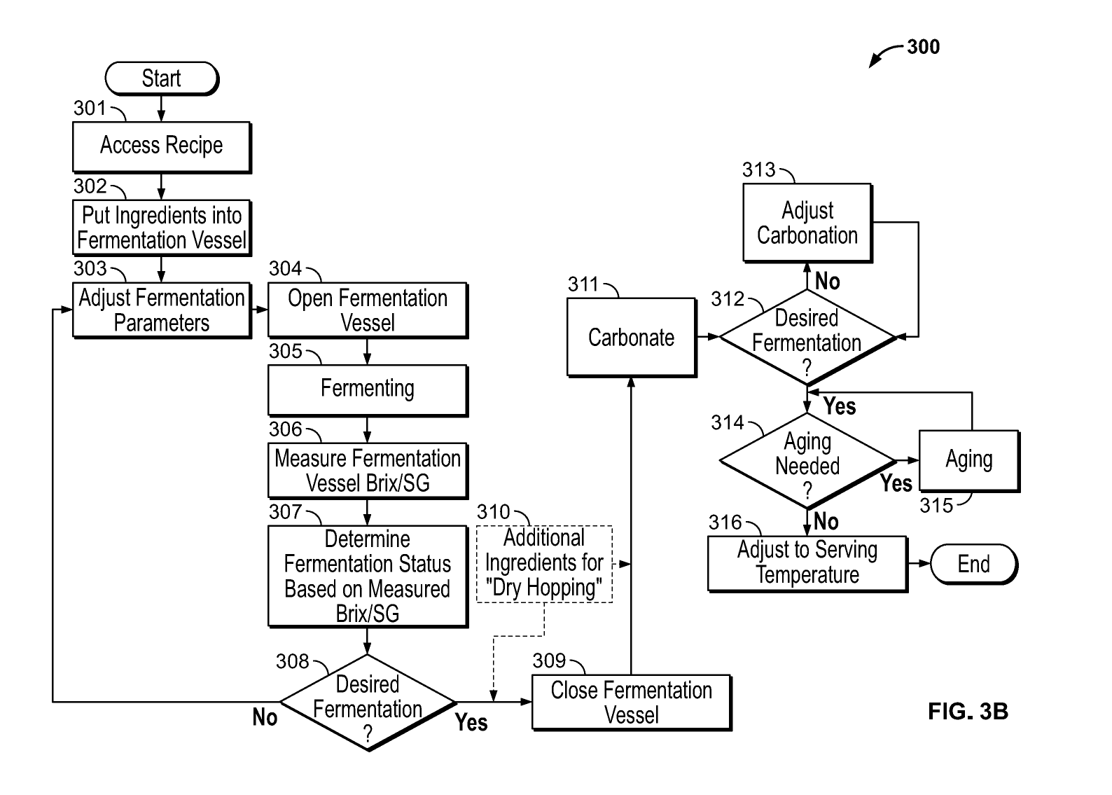

Control system 118 can include main controller 120, which may include processor 121, memory 122, storage device 123, and communication module 124. Memory 122 can include a volatile memory (e.g., RAM) that serves as a managed memory, and can be used to store one or more memory pools. In some embodiments, storage device 123 can store an operating system, security keys, brewing data, user info, and instructions for monitoring and controlling fermentation of beer, such as the methods depicted in FIG. 3B, for example. In some embodiments, storage device 123 may also store recipes for particular beers, including fermentation parameters, desired fermentation profiles, carbonation parameters, and any other suitable information for fermentation, which may be accessed and relied on in control of fermentation. Communication module 124 may allow for wired or wireless communication with any local or networked devices, including access via the internet or other network.

Control system 118 may include temperature control module 110 for controlling temperature during fermentation, carbonation, conditioning, chilling, storing and serving, pressure control module 109 for controlling pressure during fermentation, carbonation, conditioning, chilling, storing, and serving, extraction module 106 (i.e., pump 106) for extracting contents of ingredient module 104, filtration module 115 for filtering air or water entering fermentation vessel 102, dispensing module 117 for dispensing beer from device 100, sugar content(brix)/specific gravity(SG) sensor 135 for sensing sugar content or measuring specific gravity automatically in real time, pressure sensor 113 and temperature sensor 111 as described above with respect to FIG. 1A. Note that throughout this document, the sugar content/specific gravity (i.e. Brix/SG) sensor 135 may refer to, but is not limited to, Brix/SG sensor, CO₂ sensor, temperature sensor, IR sensor, and any other sensors that may be relevant in calculating brix/SG level, including ones that may utilize algorithms, formulas, or other methods to get the brix/SG level information. Although depicted separately in FIG. 1E, pressure control module 109 may include pressure sensor 113, temperature control module may include temperature sensor 111, and filtration module may include air quality sensor 129 for monitoring air entering the fermentation vessel 102.

Device 100 may include, as shown in FIG. 1E, a display module 126 coupled to controller 120. Display module 126 may include any suitable display and may display various information regarding device 100. For example, display module may display information to the user. In some embodiments, display module may display information regarding the beer type, information regarding the fermentation status such as time elapsed, time remaining, temperature, pressure, sugar content, brix, specific gravity or error indications, or any other suitable information. In some embodiments, display module may include LED indicators for providing information to the user such as an error indication, fermentation status, or other information.

Device 100 may include, as shown in FIG. 1E, an input module 134 coupled to controller 120. Input module 134 may include any suitable components for inputting information for use by controller 120 in the fermentation, carbonation, conditioning, chilling, storing, and serving processes described herein. For example, input module 134 may include a keypad or keyboard and/or buttons configured to allow the user to input recipe information or guide the processes described herein.

Device 100 may include, as shown in FIG. 1E, a mixing module 127 coupled to controller 120. Mixing module 127 may provide automatic mixing of the contents of one or more of growler 102 and pods in ingredient module 104. For example, mixing module 127 may include any suitable structure for agitating the mixture in vessel 102. In some embodiments, mixing module 127 may provide fluids such as hot water for mixing the malt-sugar contents of vessel 102.

Although not depicted in FIG. 2A, device 100 may include an identification sensor coupled to controller. Identification sensor may include an optical sensor, a wireless RFID sensor, NFC sensor or other suitable sensor that can sense a tag 5672 (FIG. 2B) including a barcode, QR code, RFID tag, NFC tag or other identifying marking or tag on the fermentation vessel, growler, or capsule. As will be described in further detail below, identification sensor may sense an identification marking and transmit a signal to controller 120 to aid in control of the fermentation, carbonation, conditioning, chilling, storing, and serving. For example, sensing of a particular identification marking may cause controller 120 to access a particular recipe associated with that marking stored in storage device 123 or accessed via communication module 124. As another example, sensing of identification marking may allow for control of the number of uses of a given fermentation capsule as well as digital rights management and/or serial number management for protection against counterfeit ingredients as described in U.S. Provisional Patent Application Ser. No. 62/385,663, filed Sep. 9, 2016, entitled Systems and Methods for Fermenting Beverages, the disclosures of which are hereby incorporated by reference in their entirety for all purposes.

As noted above, beer fermentation is the process of converting the malt sugars of wort into alcohol and CO₂, which is a key phase in achieving the desired taste for a given beer. As also noted above, each type of beer requires different fermentation conditions. In particular, brew masters have identified ideal fermentation conditions for particular types and styles of beers. In order to yield beer of comparable taste, the fermentation process must be precisely controlled under particular conditions. Deviation from these conditions early in the fermentation process may greatly affect the resulting beer, so it is necessary to continually monitor and adjust the conditions of fermentation. However, it is difficult for home brewers to emulate such conditions and continually monitor and adjust parameters at home given the inherent restrictions of traditional home brewing. Thus, fermentation of traditional home brewing is typically not precisely controlled using active feedback. Rather, home brewers typically use crude methodologies to attempt to keep generally stable temperatures and pressures over a given specified fermentation time. For example, one or two rough measurements may be made after a significant period of time to check the status, and the next phase (conditioning or carbonation) is started.

The fermentation status of a given beer is typically measured by alcohol content, which can be difficult to measure accurately. As can be understood from Gay-Lussac's chemical equation for alcohol fermentation: (C₆H₁₂O₆→2CH₂CH₂OH+2CO₂), carbon dioxide generation may also be used to ascertain a fermentation status. This is particularly so with respect to Ale brewing, where after the peak sugar consumption, which occurs after about 24 hours of fermentation, CO₂ generation levels off. The amount of sugar consumption can be determined based on the measured brix/specific gravity (i.e., SG) in the system. Moreover, the change in brix/SG can determine the current rate of fermentation which may be used to determine whether or not the fermentation is proceeding normally relative to the desired fermentation. Thus, monitoring the brix/SG of the system automatically in real time, can aid in matching fermentation of a beer to the ideal fermentation status. In view of this, automatic and precise control of fermentation will now be described with reference to FIGS. 3A and 3B.

FIG. 3A shows exemplary brix/SG and fermentation level profiles during beer fermentation according to embodiments of the invention. Specifically, graph 200 shows brix/SG 201 and fermentation level 202 plotted over time during primary (i.e., first) fermentation 204 and secondary (i.e., second or post) fermentation 205 of a given beer. In the given example, brix/SG 201 represents the ideal profile over primary fermentation 204 for a beer. Point 203 represents the time that the system was closed for measuring the pressure to determine the brix/SG, which is needed if utilizing a pressure sensor to determine the value of the brix/SG. In some embodiments of the invention, it may be desirable to measure the brix/SG at various points during fermentation and compare these measurements to the ideal profile for a given beer. In some embodiments, the comparison of the measured brix/SG data to the ideal brix profile can be used to determine the fermentation status. For example, the measured brix/SG values, the change in the measured brix/SG, and/or the rate of change of the brix/SG may be used to assess the fermentation status. In response, the temperature, pressure, or other fermentation parameters may be adjusted to speed up or slow down fermentation to match with the optimal fermentation for a given beer.

The fermentation device 100, 400 conveniently allows for the measurement of specific gravity and brix in the fermentation vessel 306. The status of the fermentation based on measured brix/SG 307 can be ascertained per FIG. 3B. The SG measurement system includes a combination of a weight sensor and a pressure sensor. The device has a known volume of wort in the growler and an RFID tag provides its original gravity (i.e., OG) information. Therefore, it is possible to convert the weight drop to a drop in SG. For example, assume the original gravity OG is 1.060 and wort volume is 3000 mL. The current SG is calculated using the following formula: [1.060−(Sum of weight drop)/3000].

For primary fermentation, a weight sensor is used and the weight is converted to SG. For the secondary fermentation, a pressure measurement system is employed. This is used because the device uses natural carbonation and there may be little if any weight drop or less weight drop due to the dissolved carbon dioxide in wort or beer. Therefore, to rely solely on the weight sensor measurement during secondary fermentation may present erroneous measurements.

By closing valve(s) it is possible to measure the current pressure since every volume of flow path, growler head space and wort volume are known. It is also possible to check how many times the valve(s) are opened and how long the valve(s) remain open. Accordingly, it is possible to convert pressure and volume of CO₂ to SG based on Gay-Lussac's equation: C₆H₁₂O₆=2C₂H₆O+2CO₂↑. Additionally, CO₂ can be converted to amount of sugar. Brix is a sugar content unit and can be converted based on the following equation:

SG≈1+(0.004×brix),

for example.

FIG. 3B shows a method for controlling beer fermentation, carbonation, conditioning, chilling, storing, and serving according to embodiments of the invention. It will be understood by those skilled in the art that the order of the steps may be switched and/or some of the steps may be combined or even skipped. The flowchart of FIG. 3B is one example of the automated fermentation process and is not intended to be limiting. Furthermore, the specific operation(s) described within each of the steps may be changed with other operations that achieve similar result. Thus, it will be understood by those skilled in the art that various other operation(s) disclosed in this application may be used instead of those shown in FIG. 3B. The process will now be described with reference to FIG. 3B and other corresponding figures.

At step 301, a recipe for a given beer may be accessed by controller 120. In some embodiments, the recipe may include a desired brix profile for the desired beer to be made. As described above, controller 120 may access a given recipe based on identification of a particular fermentation growler by identification sensor 128. For example, identification sensor 128 may identify the type of beer by sensing an RFID tag on fermentation capsule and may access an internally or externally stored database with the recipe corresponding to the type of beer identified. Alternatively, controller 120 may access a given recipe based on user input via input module 134 or communication module 124. Recipes may include any suitable information for fermentation, carbonation, conditioning, chilling, storing, and serving of a given beer, such as prescribed timing, rates of fermentation, temperatures, pressures, desired CO₂, or other information.

At step 302, controller 120 may actuate the components so as to put ingredients from vessel 102 and ingredients module 104 together into vessel 102. Controller 120 may actuate components according to any instructions provided in the recipe accessed at step 301. For example, controller 120 may actuate extraction module 106 to pump air through ingredients module 104, causing the contents therein to be mixed with the contents of vessel 102. As another example, controller 120 may actuate mixing module 127 to aerate and/or agitate the contents of capsule 102 prior to causing the contents of ingredient module 104, so that the yeast has enough oxygen for fermentation. Controller 120 may also actuate any of valves 125 in order to allow mixing of the relevant ingredients. As will be described below with respect to step 314, in some embodiments, controller 120 may apportion the amount of ingredients extracted from a given capsule to allow for additional ingredients to be extracted at a later time in processing according to a recipe for a given beer.

At step 303, controller 120 may initialize fermentation parameters based on the recipe accessed in step 301. For example, controller 120 may set temperature control module 110 to a desired starting temperature and/or pressure control module 109 to a desired starting pressure based on the recipe for a given beer.

At step 304, controller 120 may actuate valve 114 into an open position to allow open fermentation to occur. Controller 120 may keep valve 114 in an open position for a predetermined amount of time based on the given recipe for a beer. In some embodiments, the initial predetermined amount of time for open fermentation may be determined based on the recipe accessed in step 301. For example, for a given beer, the predetermined amount of time for open fermentation may be determined to be a particular order of magnitude less than the time expected for complete fermentation to occur to optimize monitoring and control of fermentation relative to a desired profile.

At step 305, fermentation may begin under the fermentation parameters set at step 303. At step 306, while in open fermentation, controller 120 may receive a brix/SG measurement from brix/SG sensor 135. In some embodiments, controller 120 may receive multiple brix/SG measurements from brix/SG sensor 135. For example, brix/SG sensor 135 may measure a sugar consumption, a change in brix/SG over a period of time, and/or the rate of change in pressure and send these measurements over time to the controller 120. In some embodiments, controller 120 may close valve 114 briefly in order to receive accurate pressure and/or brix/SG measurements.

At step 307, controller 120 may determine a fermentation status based on the received brix/SG (and/or pressure) measurement or measurements. For example, controller 120 may determine the fermentation status based on the brix/SG value. As another example, controller 120 may determine fermentation status based on the brix/SG and the rate of change of the brix/SG during open fermentation. The fermentation status may correspond to a level of brix/SG, which may in turn correspond to an alcohol content level. In some embodiments, controller 120 may compare the brix/SG measurement or corresponding level of alcohol to a fermentation level as accessed from the relevant recipe to determine the fermentation status. In some embodiments, controller 120 may compare the rate of change of the brix/SG to a desired rate of change given from the relevant recipe to determine the fermentation status. In some embodiments, controller 120 may calculate the brix/SG value from rate of change in pressure to determine the fermentation status.

At step 308, controller 120 may determine whether the desired fermentation has been reached based on the fermentation status determined in step 307. If a desired fermentation has been reached, the controller 120 may proceed to step 309 to close the vessel and then to step 311 and beyond to carbonate and/or condition the fermented beer. If a desired fermentation has not been reached, the controller may repeat steps 303-308 as necessary, or as dictated by the recipe for the relevant beer. For example, if the fermentation status determined in step 307 indicates that the rate of fermentation is too slow, the controller 120 may increase the temperature set by the temperature control module 110 to stimulate fermentation. Alternatively, if the fermentation status determined in step 307 indicates that the rate of fermentation is too fast, the controller 120 may decrease the temperature set by the temperature control module 110 to slow fermentation down. Then, controller 120 can proceed through steps 303-308 until the desired fermentation is achieved. Thus, precise and automatic control of fermentation relative to the desired fermentation profile may be achieved.

In some embodiments, prior to or after transitioning the fermentation vessel to a closed fermentation state for carbonation at step 309, optional step 310 may include adding additional ingredients to fermentation vessel 102 to achieve the desired flavor, aroma and/or color for a given beer. In particular, it will be understood that while open fermentation aids in releasing undesirable off flavors generated during the first fermentation, some desirable flavors and/or aromas may also be released during open fermentation. As a result, in order to replenish or otherwise achieve a desired flavor or aroma, brewers employ various “dry hopping”, flavoring and coloring techniques to add flavors, aroma and/or colors later in the beer processing. Since processing after primary fermentation is primarily under a closed fermentation state as noted below, it is desirable in some embodiments to automatically or otherwise extract additional ingredients prior to or after the transition to closed fermentation. Thus, it will be understood that controller 120 may actuate automated dry hopping in some embodiments. Accordingly, in some embodiments, controller 120 may cause additional ingredients such as hop flavoring to be extracted from any of pods from ingredient module 104 prior to transitioning to closed fermentation/carbonation. In some embodiments, controller 120 may automatically extract additional ingredients from a designated pod or pods from ingredient module 104 based on the recipe for a given beer. For example, controller 120 may extract a particular amount of hop oil or hop pellets from pods in ingredient module 104 at step 302, and extract any remaining amount of hop oil or hop pellets from pods in ingredient module 104 after it determines that the desired primary fermentation is completed, but before or after it transitions to a closed fermentation state at step 309. In some embodiments, controller 120 may prompt a user to add additional ingredients to one of pods in ingredient module 104 via display module 126 or remotely via communication module 124. In some embodiments, controller 120 may actuate dry hopping module 105 which may siphon the wort to a portion of dry hopping module 105 that contains dry hops, flavors, colors or any other ingredients to extract the flavor, aroma and/or color of the ingredients to the siphoned wort to achieve the taste, aroma and color of the desired beer.

At step 311, carbonation may be initiated. In some embodiments, carbonation may be achieved by generation and capture of CO₂ during a second fermentation with the fermentation vessel in a closed fermentation state. It will be understood that natural carbonation by this secondary fermentation may provide the advantage of improved control of the taste as compared to using additive sugar or forced carbonization as is typical in home brewing. In some embodiments, carbonation may be controlled by opening and closing of a valve of the fermentation vessel such as valve 114 in device 100. In some embodiments, controller 120 may receive a measured pressure from pressure sensor 113 and control the opening and closing of valve 114 based on a desired CO₂ level for a given beer to achieve the desired carbonation. For example, if the pressure indicates a current CO₂ level is too high, controller 120 may open valve 114 to vent off pressure. It will be understood that automated control of valve 114 may provide more accurate and easier carbonation than imprecise manual valve and pressure gauge control that are employed in home brewing. In some embodiments, based on the desired CO₂ level, controller 120 may close valve 114 slightly prior to the completion of all fermentation based on the fermentation status such that the remaining CO₂ generated by additional (secondary) fermentation provides the requisite amount of carbonation. Although described in terms of carbonation, it will be understood that automatic control of valve 114 may be beneficially employed during any phase described herein, including but not limited to fermentation, carbonation, conditioning, aging, chilling, storing, and serving.

In some embodiments, vessel 102 may be removed from housing 101 for carbonation at another location. For example, when it is determined that the fermentation status is such that remaining CO₂ generated by additional (secondary) fermentation provides the requisite amount of carbonation, controller 120 may output a notification to the user for optionally being able to remove vessel 102 and its contents to another location for chilling and final carbonation. It will be appreciated that allowing for carbonation and storage elsewhere will free device 100 to commence fermentation of another fermentation vessel 102. In other embodiments, however, carbonation may also be finished within device 100 as described above.

At step 312, if carbonation is finished within device 100, it is determined based on the relevant recipe whether additional fermentation is needed. For example, for particular beer recipes, secondary or further fermentation may be required. If additional fermentation is needed, step 313 is repeated with reference to the recipe for such additional fermentation as shown in step 313 until the desired fermentation is achieved as shown in step 312.

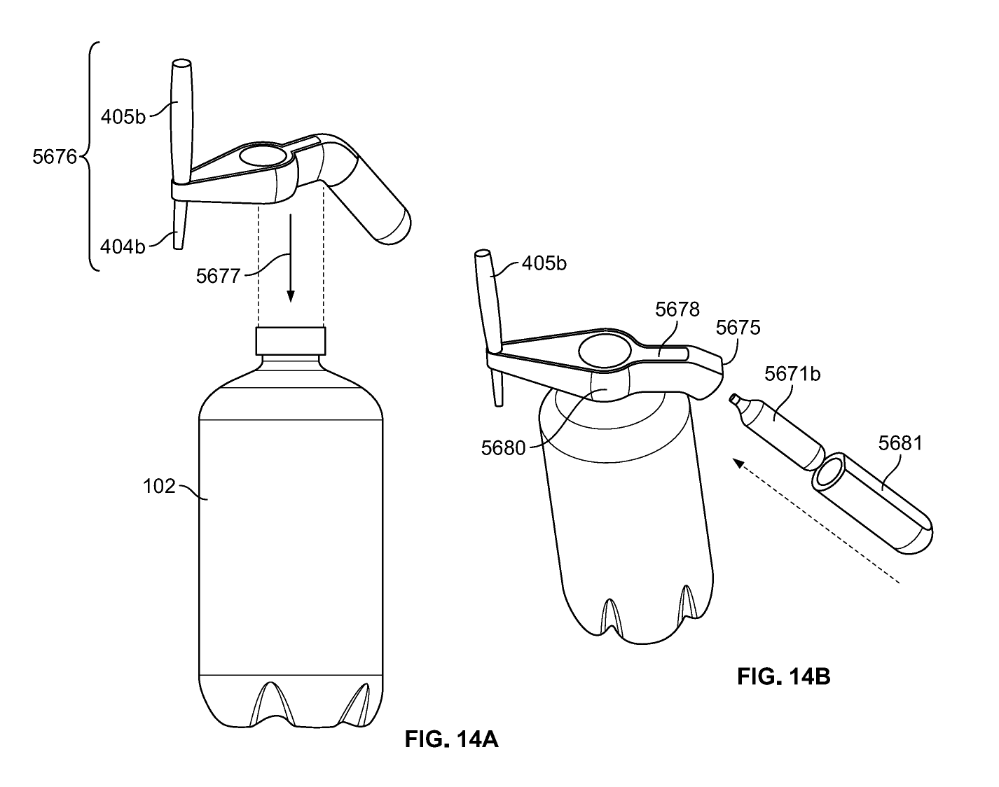

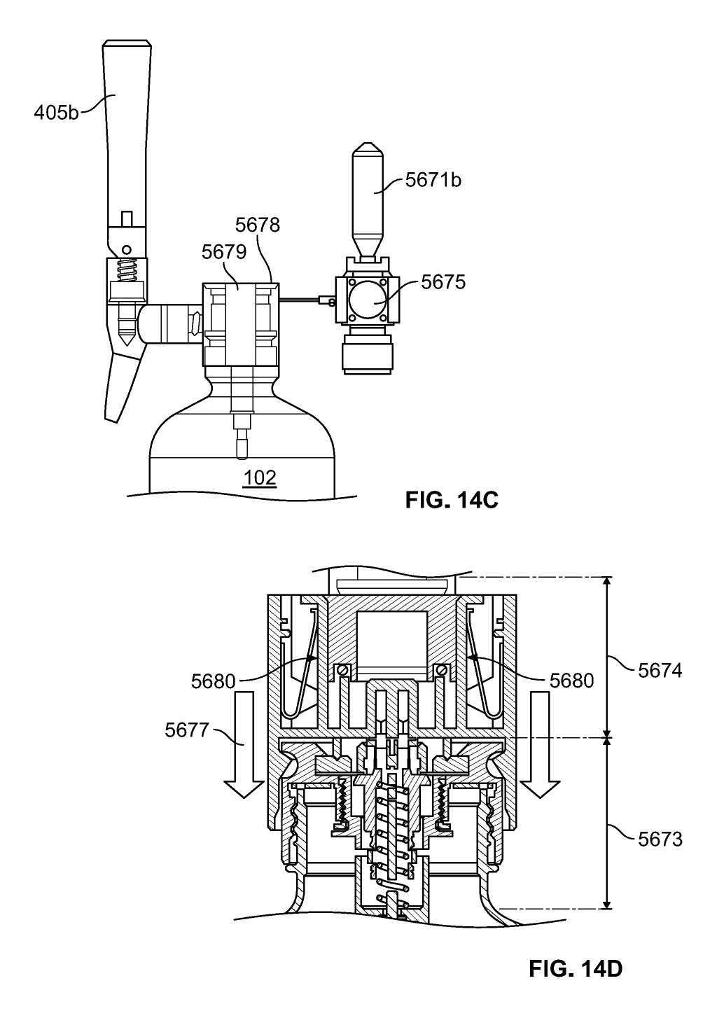

Once additional fermentation is not needed, it is determined, based on the relevant recipe accessed in step 301, whether aging is required, as shown in step 314. If the beer recipe requires further aging, the controller 120 may perform the aging as shown in step 315. Aging may include adjusting the temperature setting of the temperature control module 110 to the desired temperature for aging of the beer as defined in the recipe and maintaining the beer at the desired temperature for a predetermined amount of time or until the desired aging is achieved. In some embodiments, the predetermined time and/or the desired aging requirements for the brewed beer may be defined in the recipe. If aging is not required or aging step 315 is completed, controller 120 may adjust the temperature setting of temperature control module 110 to the desired serving temperature of the beer as shown in step 316, where the desired serving temperature value for the brewed beer may be defined in the recipe obtained in step 301. For example, barley wines and imperial stouts are best served at temperatures around 60-64° C., most ales are best served at temperatures around 53-56° C., and most lagers are best served at temperatures between about 43-46° C. In some embodiments, controller 120 may alert the user via display module 126 (including LEDs), sound, and/or an outside device, such as a smart phone 5670, via module 124, and/or any other means of communication to the user, that the beer is ready to dispense via dispensing module 117. Alternatively, the user may remove vessel 102 from the fermentation device post fermentation for storage and/or for mobile dispensing per FIGS. 14A-14D. This allows the user to place a second fermentation vessel in the device 100, 400 to ferment a second beverage.

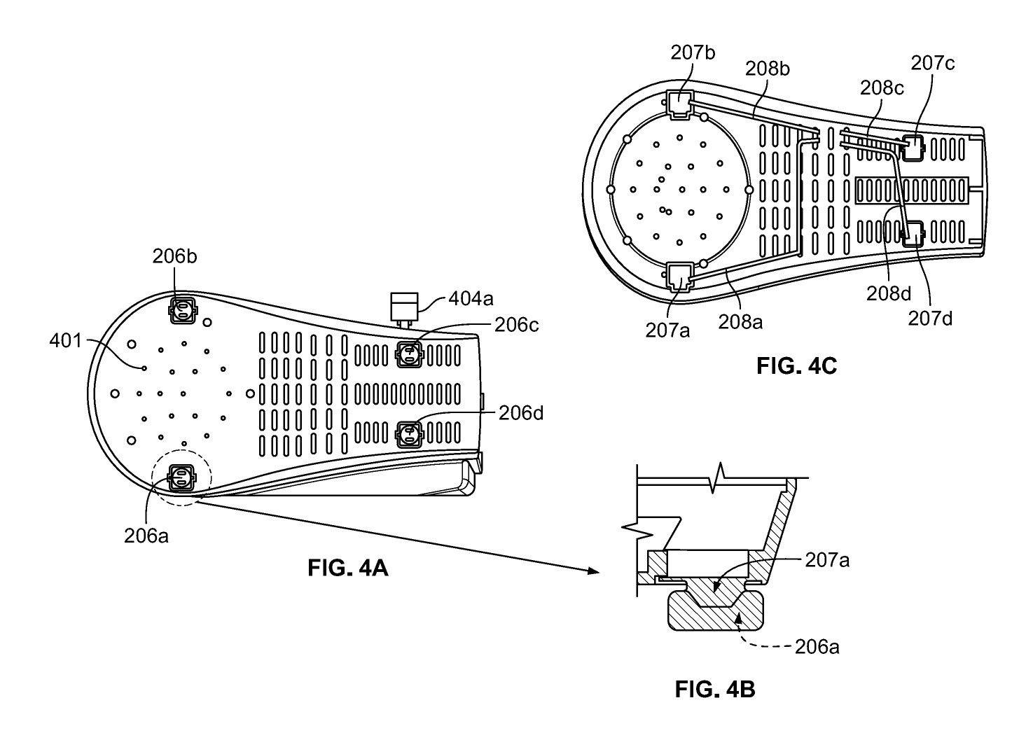

FIGS. 4A-4C show exemplary load cell embodiments. As a real-time fermentation measurement method, a method of measuring SG in real time using a load cell to sense weight can be used. The load cell(s) may be located under the fermenter inside the unit or at the bottom of the unit, for example. As shown in FIGS. 4A-4C, each load cell includes a rubber foot 206a, 206b, 206c, 206d and also a load sensor 207a, 207b, 207c, 207d. Each load sensor is connected to the controller via a corresponding connection 208a, 208b, 208c, 208d.

When the load cell is located in the bottom of the fermenter inside the device, a structure is formed in which the fermenter can support the weight of the device. Initially, only the weight of the grower is measured, so the accuracy may be high, but there may be an error depending on the fixed part of the fermenter. Since the load cell is located at the position inside the device, it receives less external influence.

If it is located on the bottom of the appliance, it is located in a position where it can support the whole appliance. It may be measured with a 4-point support (FIG. 4A) similar to a general scale. Since it measures the total weight, it is influenced by the outside. Therefore, an algorithm is supplied to eliminate external influences.

Initial volume and original gravity are required to calculate the SG through the weight sensor. This can be obtained from the information recorded in the tag (e.g., QR code, bar code, RFID tag, or NFC tag) on the fermentations vessel, growler, or container. The change in specific gravity can be calculated from the initial volume and the weight reduced in original gravity (i.e., OG). For example, if the initial specific gravity is 1.060 and the volume of the container is 3000 mL, the initial weight is 3180 g. A change in specific gravity of 0.001 results in a weight change of 3 g. This gives OG-reduced weight/volume (mL)=current SG. Measuring the weight may cause errors due to the influence of external vibration, wind or something placed on the device. In order to exclude this error, the mean value of the weight is measured at intervals of time, and when this value stabilizes, the point is balanced to zero. When the average value of the constant time is decreased based on this value, the sum of the values is added to obtain the sum of the reduced weights. When fermenting beer, the specific gravity changes from about 1.060 to 0.02 per day, so it changes only about 60 g per day and 2.5 g per hour. Therefore, the weight change beyond this range is removed through the band filter, and when this deviation occurs, zero balancing again increases the accuracy of the weight measurement. Also, since the influence of disturbance to the surrounding environment may be reduced during nighttime, the sum of the reduced weight during one day (or several hours) may be corrected based on the measured value at this time. Since the weight is continuously added to the average value over a certain period of time, it is possible to eliminate the error by correcting the decrease in the weight.

The final gravity (i.e., FG) measurement may not reach the FG in terms of weight due to the influence of the amount of dissolved CO₂ when performing the natural carbonation when measured by weight. According to the yeast input in the recipe, the attenuation (i.e., percentage of final fermentation) can be obtained and it is possible to estimate the FG value. When the target carbon dioxide pressure set in the recipe is reached, a solenoid valve is opened to maintain the target pressure and carbonation. When the valve reaches the target pressure and the valve does not open for a certain period of time or when the target pressure is not reached, and the number of openings of the valve is measured, it is possible to know the completion time of the final fermentation by converting into FG. For example, if the valve does not open for a certain time after reaching the target pressure, the expected FG value is derived according to the yeast attenuation. If the target carbon dioxide concentration is too low to reach the target pressure, the expected FG value is calculated according to the yeast attenuation, or the CO₂ pressure reached at the SG at the time of natural carbonation is calculated as the amount of generated CO₂ FG can be calculated. Similarly, it can be measured through the number of open valves. Depending on the pressure/valve open time, the amount of CO₂ discharged can be taken from a measured or pre-calculated value or equation. It is also possible to calculate the amount of total sugar consumed in natural carbonation by calculating the amount of CO₂ from the target pressure and converting it into the amount of sugar consumed and converting the amount of carbonated material into the amount of consumed sugar.

The fermentation device tracks the pressure (P) from the pressure sensor, volume (V) based on the fixed head space and flow paths, temperature (T) from the temperature sensor, and value (R) which is a constant. Based on these values and relationships, it is possible to determine (e.g., calculate) n of CO₂ (mole number) using the ideal gas law formula: PV=nRT. Additionally, using the empirical formula: C₆H₁₂O₆→2C₂H₅OH+2CO₂↑, we find that 180 g of C₆H₁₂O₆ produces about 88 g CO₂ and the expected yield is about 94%, so 82.72 g of CO₂ gas will be generated. It is noted that 82.72 g of CO₂ can convert to about 180 g sugar. Sugar weight is [n mole×44 g CO₂/1 mole×180 g sugar/82.72 g CO₂=95.74×n (or 90×n)], so it is possible to use the empirical formula as a lower limit value and the ideal gas law formula as an upper limit value. Thus, SG will be [present specific gravity−(from 95.74×n to 90×n)/initial wort volume]. The initial wort volume is a given value but it can change from one brewing session to another depending on the type of beer being brewed, for example. The growler has the determined volume of wort which can be tracked by the device via data on the tag, for example. The current SG value can be calculated by using the following relationship: SG=OG−(weight change/initial wort volume)−(90 n to 95.74 n)/initial wort volume. Additional conversion information for brix (i.e., the sugar content of an aqueous solution), SG (i.e., the ratio of the density of a substance to the density of a standard) and Plato (i.e., an empirically derived hydrometer scale to measure density of beer wort in terms of percentage of extract by weight), include: specific gravity (SG)=1+(0.004×brix); specific gravity=1.000019+brix (0.003878634261280); brix=(SG−1.000019)/0.003878634261280; SG to Plato=(258.6−(258.6/SG)); and Plato to SG=(((182.4601*SG−775.6821)*SG+1262.7794)*SG−669.5622).

In this way, the change in specific gravity can be obtained in real time throughout the entire process of fermentation, including brewing beer. It is also possible to understand the progress of the fermentation process and the current state of fermentation. In addition, automated fermentation can be achieved by making the necessary decisions during the fermentation process or according to a programmed recipe.

The user can confirm a previously entered recipe and information obtained through progress of fermentation, and the device can automatically perform beer fermentation based on the information and provide the user with beer of the same enjoyable taste as a craft brew. Certain embodiments also allow the user to adjust parameters to create a beer personalized to the preferred taste of the user.

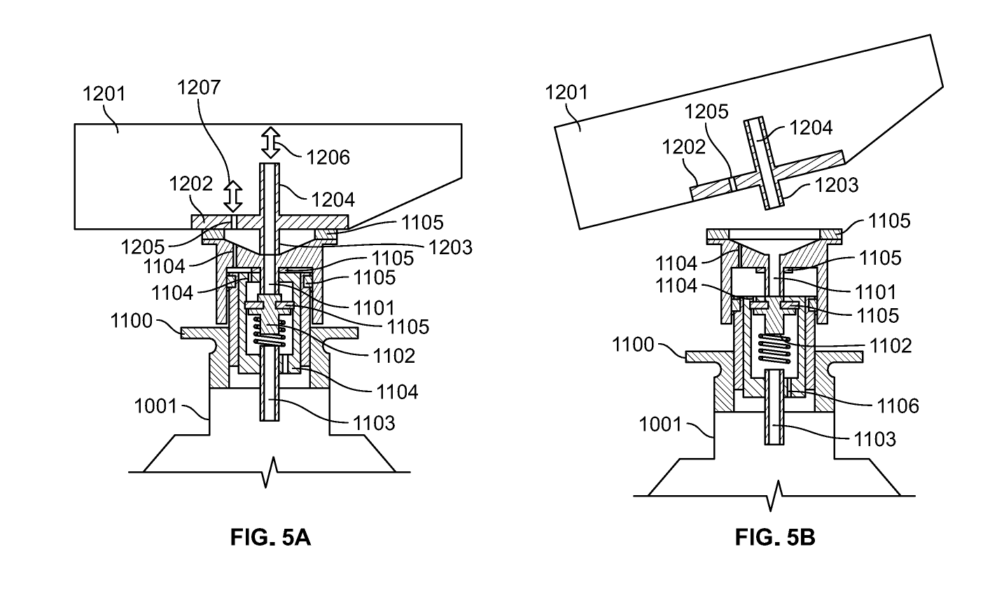

The operation of cap assembly 1100 will be further described with respect to FIGS. 5A and 5B. FIG. 5A shows the interaction between cap assembly 1100 and cover 1201 of a beer fermentation device when a growler 1001 is installed and cover 1201 is closed, and FIG. 5B shows cap assembly 110 and cover 1201 when cover 1201 is opened, in accordance with embodiments of the invention. As can be seen in FIG. 5A, cover 1201 may have a fermentation growler interface 1202 that includes a protrusion 1203, a beer channel 1204, and an air channel 1205. It will be understood that beer channel 1204 may correspond to the channel feeding dispenser 117 shown in FIG. 2A and air channel 1205 may correspond to the channel filtered by filtration module 115 to allow open fermentation shown in FIG. 2A. As can be seen in FIGS. 5A and 5B, cap assembly 1100 may include fluid channels 1101 and 1103 which may be in communication with each other depending on the position of spring assembly 1102, air flow channels 1104 which may be open or closed depending on the position of spring assembly 1102, and sealing components 1105 which may ensure the proper seal between chambers as desired.

As shown in FIG. 5B, when cover 1201 is rotated to an open position, protrusion 1203 is not engaged with cap assembly 1100. In this position, spring assembly 1102 is at an elevated position which keeps fluid channels 1101 and 1103 sealed from one another and similarly keeps channel 1104 sealed. It will be understood that spring assembly 1102 may be biased in this position such that the contents of growler 1001 are sealed by default, and only unsealed when cover 1201 and protrusion 1203 engage with cap assembly 1100. Upon closing of cover 1201 into the position shown in FIG. 5A, it can be seen that spring assembly 1102 may be lowered by the engagement of protrusion 1203 with cap assembly 1100. As can be seen in FIG. 5A, when spring assembly 1102 is lowered as shown, channels 1101 and 1103 may be in fluid communication with one another. In this position, it can also be seen that channel 1204 may be in communication with 1101, such that contents may flow between the device and growler as shown by arrows 1206 and as needed to add ingredients and/or distribute the beer as described above. Similarly, it can be seen that when the spring assembly 1102 is in the lowered state shown in FIG. 5A, fluid channels 1104 may also be opened to allow air flow between the device and growler as needed for open fermentation as described in detail above.

It will be understood that cap assembly 1100 and its interaction with fermentation growler interface 1202 of cover 1201 may have benefits of allowing a user to remove the fermentation growler (for example, after primary or secondary fermentation is complete) and maintain the desired sealed environment in the fermentation growler to keep the beer at the desired quality during storage. In contrast, devices which only seal the vessel when the cover is closed do not allow maintenance of the environment of the contents when the cover is open and thus removal of the fermentation vessel is not possible, or may deteriorate the beer quality.

Conventional keg connectors employ a check valve system in the keg which prevents extra beer from spilling out when the keg is “tapped” (i.e., coupled to the dispenser). However, an upper coupler typically does not have a check valve system and remains open. This may cause the beer to spill and/or allow contaminants to enter the fermentation vessel and fowl the beer.

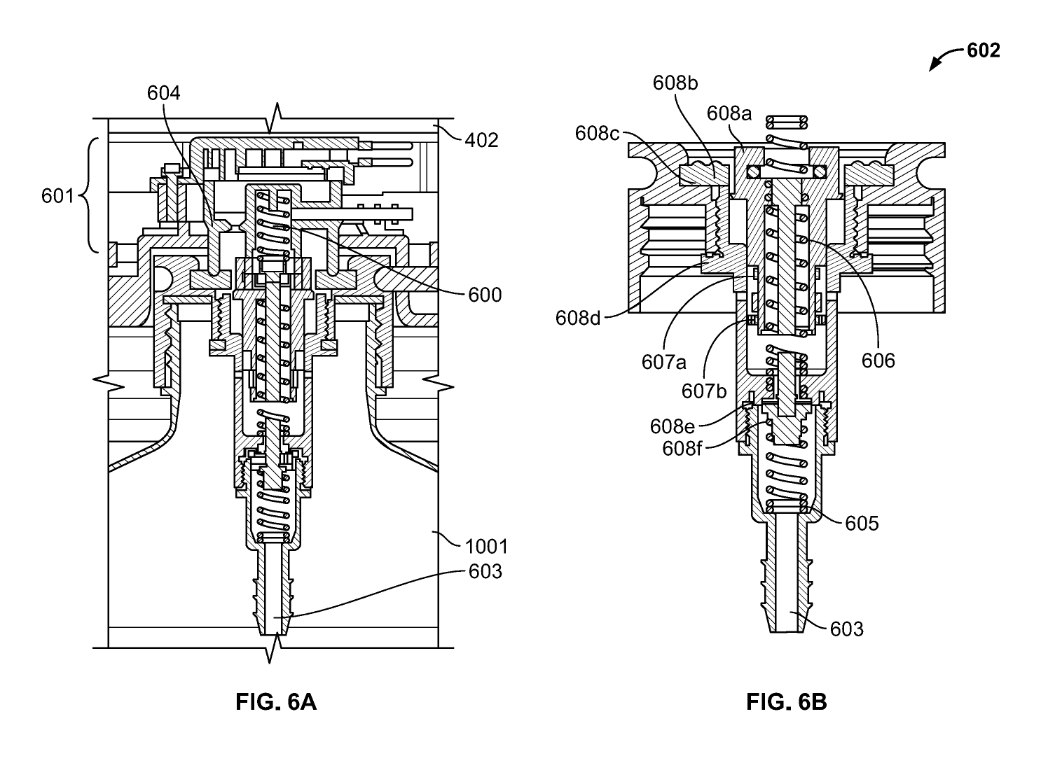

In addition to the cap assembly 1100 and cover 1201 described with respect to FIGS. 5A-5B, the cap 601 of FIG. 6A may also include a check valve 600. FIG. 6A shows the interaction of a locking structure 602 (isolated in FIG. 6B) between a closed cap assembly 601 and an closed main cover 402 of a portable fermentation device 400.

FIG. 6B shows a locking structure detail of FIG. 6A with the main cover 402, cap assembly 601, and growler 1001 removed. As shown in FIG. 6, the addition of a cover check valve 600 offers additional advantages over the beer channel 1204 (without a check valve) in FIGS. 5A and 5B. For example, it prevents unexpected droplets of beer from spilling when the cap and cover 402 are disconnected. In this manner, beer spray and foam do not escape and fowl the inside of the cover. This connection assembly is designed such that it cannot be connected if the cap is not mounted exactly in the proper position or orientation. The cap 601 and locking structure 602 are pushed together which, in turn, pushes bottom spring 605 and upper spring 606 together. This opens the beer line 603 and air/CO₂ flow paths 604 and helps users confirm the connection is secure. A series of lip seals 607a, 607b and O-rings 608a, 608b, 608c, 608d, 608e, 608f maintain proper seals throughout the beer 605 and air flow 604 pathways.

Embodiments of the subject invention include connecting the growler to the flow path of the fermentation device by inserting a growler on the device body and closing the top lid. In this case, the holding structure inside the fermentation vessel of the fermentation device is always designed to fit the bottom of the grower so that the connecting point always keeps a constant and consistent position with respect to the growler or other fermentation vessel. Additionally, when the cooler is operated, the holder is perforated to form and distribute air through a circulation path. Through the holes, it is possible to expect the contact time that the air can sufficiently contact the growler, so that the heat exchange and performance thereof can be improved.

An air flow path and a beer flow path are connected to the lid portion of the fermentation device, and a check valve system is installed in the beer flow path. This can prevent the beer from splashing and releasing the pressure of the appliance even if the lid is accidentally opened. Also, when the grower is installed and removed, the remaining beer flows to prevent the surroundings from becoming dirty and potentially contaminated. It is possible to maintain the entire flow path in the closed state and minimize the influence airborne microbes on the fermentation process.

FIG. 7A is a diagram of a dry hopping system 400 according to embodiments of the invention. Referring first to FIGS. 7A and 7B, the system 400, 105 works by placing a pod 5699 in the pod holder 5682 as shown by direction 5702. The pod may be made from any suitable material for containing liquids and foods, including but not limited to polyethylene terephthalate (PET), polypropene (PP), polyethylene (PE), or any other similar suitable materials. The pod 5699 may contain a yeast pod, a hop pod, a flavoring pod, a color pod, and/or any other ingredients.

A dry hopping bag 5700 is added in the general direction 5701. The bag 5700 may be made of a mesh material (similar to tea bag packaging) and is wrapped again with a material such as EVOH film, an aluminum laminated film, or similar material that may expand as ingredients are added and remain durable and sealed during fermentation, carbonation, conditioning, aging, chilling, storing, and serving of the beer.

When at least some of the wort in the growler 102 has fermented to beer 5703, about 200-300 ml of beer 5703 is then siphoned from the growler 102 to the dry hopping tank 902 via peristaltic pump 5695. A level sensor 913 monitors the level of beer 5703 so it does not overflow, for example. After the beer 5703 has had some time to infuse with the hops contained in the hopping bag 5700 (now submerged in the beer 5703) and mix with the pod 5699 dispensed into the beer in direction 5704 (FIG. 7D), the beer 5703 is returned to the growler 102. This process of transferring the beer from the growler to the dry hopping tank and back can be repeated any number of times depending on the programmed recipe or the intensity of flavor and other characteristics desired by the consumer. The amount of time the beer contacts the pod and hopping bag is also adjustable. Generally, the long the time in contact, the stronger the beer. Some beers may not require the hopping bag, for example. Different pods can be inserted into the pod holder during the process as well. Accordingly, many combinations are possible.

FIG. 7E is a diagram showing degradation prevention as related to FIG. 7A. If the contents (e.g., pod and hopping bag) placed the dry hopping tank 902 are exposed to air for a period of time, their characteristics may degrade due to oxidation. Oxidation reduced the freshness and flavor. Oxidation is significantly reduced, and sometimes even eliminated, by pumping CO₂ gas 5705 into the dry hopping tank 902 in direction 5706. As beer ferments, CO₂ gas is generated and accumulates in the dry hopping tank 902 via a flow path give the appropriate configuration of valves. Because CO₂ is heavier than air, it is filled from the bottom of the dry hopping tank 902, and the excess CO₂ is discharged through the top air filter 5688c. The hopping bag 5700 is located at the bottom of the dry hopping tank 902 and the entire tank is sealed and no air can enter. Air may be introduced through an upper air filter, but the CO₂ concentration in the module is maintained above 90% because CO₂ is continuously released during fermentation. It may also be possible to use CO₂ from a cartridge 5707 in the unlikely event that insufficient CO₂ is produced from fermentation alone.

The pod 5699 is located at the upper part of the tank 902 so that the influence of air contact on the lower part where the hops are located inside the pod is minimized. Even if the pod extract is injected, it can be purged with CO₂ that is still produced even if a small amount of air comes into contact.

This is the same for beer fermented during dry hopping. When dry hopping or additional flavor is added, the beer is transferred to the dry hopping tank 902. In this case, the inside of the module maintains more than 90% CO₂ concentration, which minimizes the amount of deterioration due to oxidation.

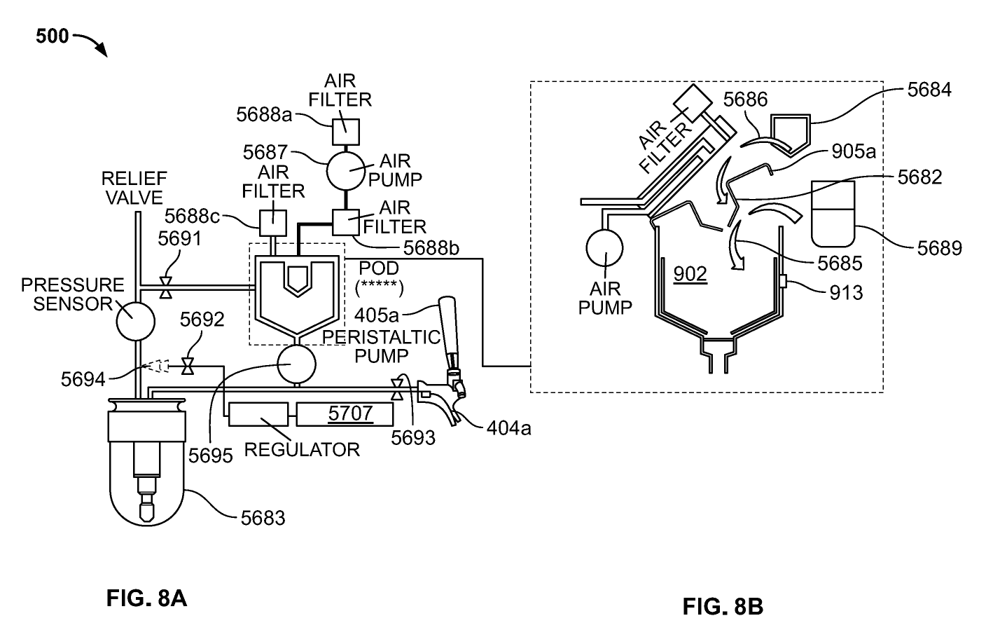

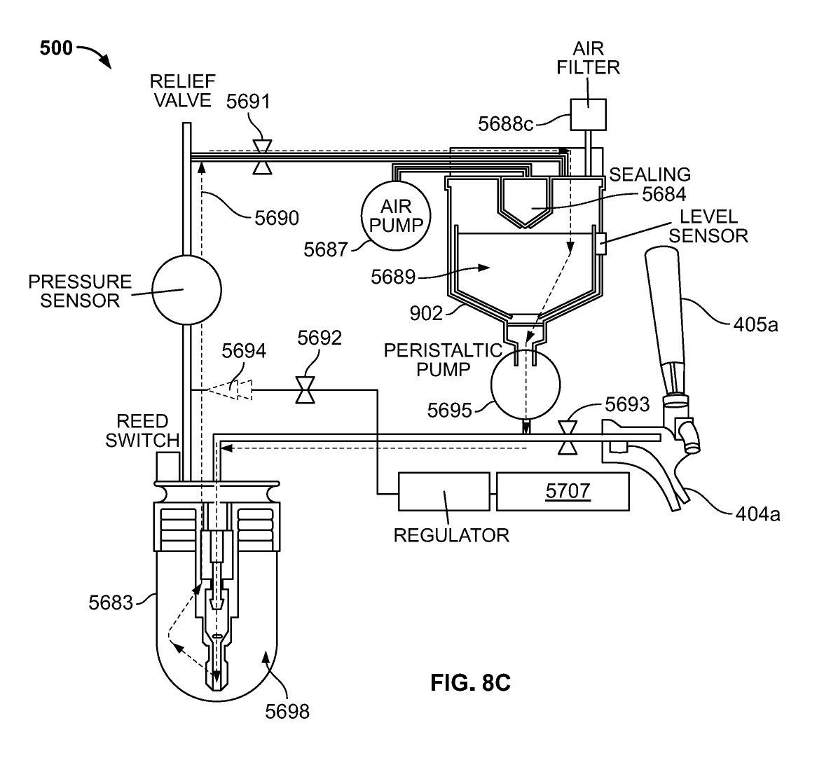

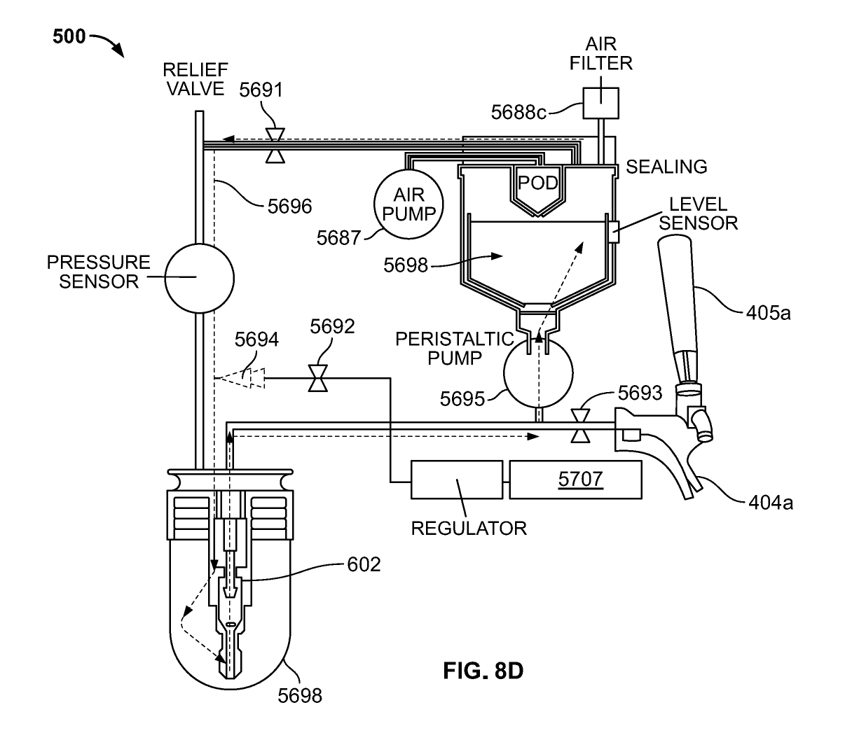

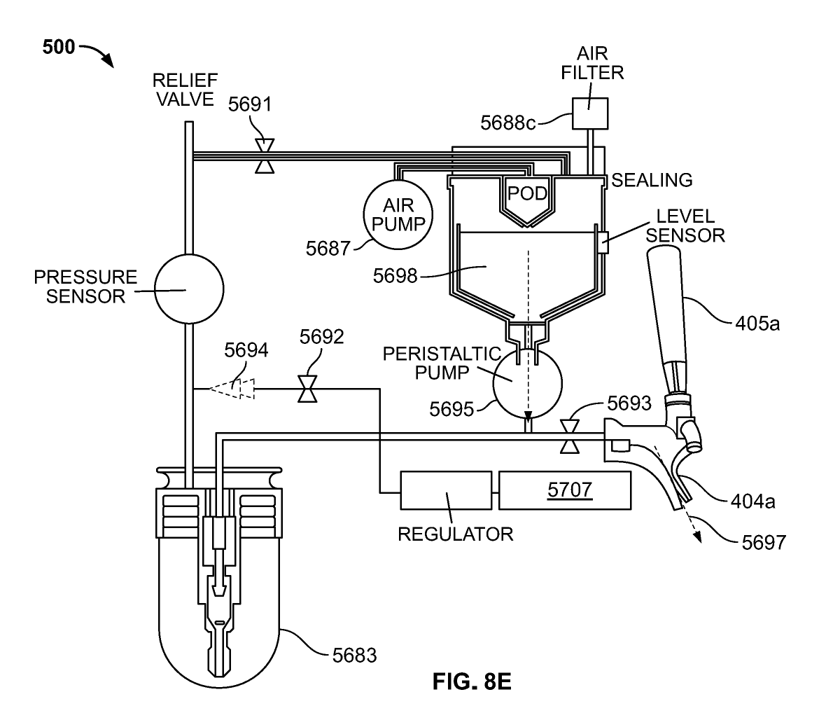

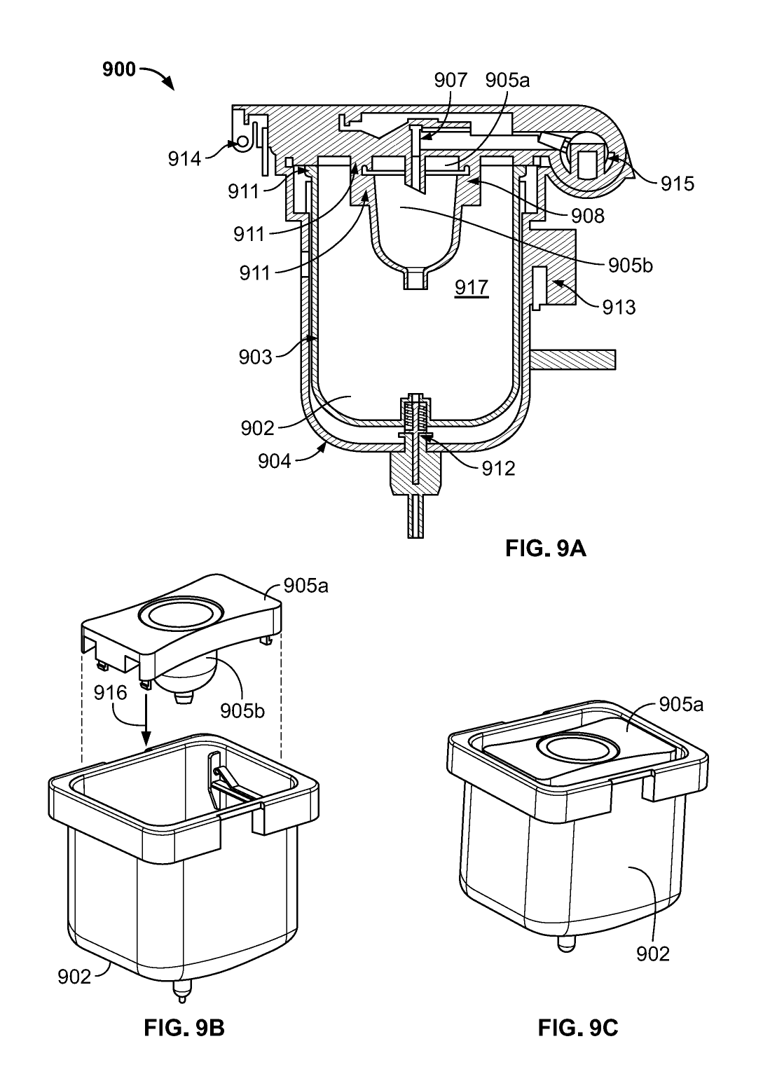

FIGS. 8A-8E relate to an automatic cleaning of the fermentation system. Sanitizing is very important when preparing fermented beverages. If surfaces and flow paths are not regularly kept clean, a host of contaminants (e.g., bacteria, biofilms, dust, viruses) can quickly cause foul odors, bad tastes, and other problems that may ruin the fermented beverage or cause illness, for example. It is understood that the sanitizing solution used to clean the fermentation system may be a food grade cleaner that would ideally not require flushing or rinsing with water. Some examples of such cleaners may include, but are not limited, to citric compounds, vinegar, potassium hydroxide, and sodium hydroxide. The fermentation vessel 406 (FIG. 1D), top lid 905a (FIG. 9A), separable dry hopping tank 902 (FIG. 9A), sanitizing cap 5683 (FIG. 8A), pod position holder 5682 (FIG. 8B), and other components may be separately removed from the fermentation device for individual cleaning.

To begin the cleaning protocol, the fermentation vessel is removed and replaced with a sanitizing cap 5683. Other separable components may be optionally removed and cleaned separately. Next, as shown in FIG. 8B the top lid 905a is opened, and a volume of water 5689 is poured into the separable dry hopping tank 902 in direction 5685 and a sanitizing pod is placed in the pod holder 5682 in direction 5686. The sanitizing pod contains a sanitizing (i.e., cleaning) agent. The top lid 905a is closed in an opposite direction of 5686.

FIG. 8C depicts the cleaning system loop 500. When the air pump 5687 is activated, air is pushed through the system and the sanitizing agent moves into the dry hopping tank 902. The water inside the tank mixes with the sanitizing pod to release the sanitizing agent into the water. Alternatively, a sanitizing agent may be dissolved in the volume of water 5686 and then the solution may be poured into the dry hopping tank 902 thus eliminating the need for a sanitizing pod 5684. The initial flow of sanitizing solution (sanitizing agent and water) is in a clockwise direction 5690. Several air filters 5688a, 5688b, 5688c prevent contaminants from entering the device 100, 400 while the air pump 5687 is working. The pod position holder is also cleaned and sanitized.

When cleaning is activated, an algorithm controls sanitizing the system. The cleaning algorithm is set by adjusting the circulating flow rate, circulation time, immersion time, and flow direction (i.e., clockwise, counterclockwise). The counter clockwise direction (FIG. 8D) is performed after clockwise direction (FIG. 8C) to increase the cleaning effect through the flow velocity and shear flow in the flow path. As shown in FIG. 8C, valves 5691, 5692 are open and valve 5693 is closed. Valve 5693 may be a mechanical valve associated with faucet 404a, for example.

A combination of flow rate and submerged time in contact with cleaning agent maximizes the efficient cleaning and sanitizing of the system such that the complex interior of the connecting cap 5683 and the flow paths of the fermentation device 100, 400 are thoroughly cleaned without disassembling the device.

During cleaning, the CO₂ flow path is blocked by a check valve 5694. When completing the circulation loop, the contact portion of the check valve 5694 is also cleaned.

It is possible to increase the effect of cleaning through the shear flow rate rather than simple immersion. A special sanitizing cap 5683 is required for this purpose. When cleaned, the locking structure (FIG. 6B) is attached to the cap 5683 in place of the fermentation vessel. The beer line 603 is located at the bottom of the sanitizing cap 5689 (FIG. 8C) so that no residue is left when the cleaning operation is complete. In this way, the beer line 603 and air/CO₂ flow paths 604 are cleaned. A peristaltic pump 5695 operates at valve 5691 open and valve 5693 closed to clean the flow paths in a clockwise direction 5690 where it is otherwise difficult to clean without removing the cap, for example.

FIGS. 8D is a diagram of the cleaning system loop with flow in a counterclockwise direction 5696. It is contemplated that the order of the flow direction (i.e., clockwise and counter clockwise) can be reversed (counter clockwise and clockwise) by the algorithm. The change in direction is important because the swishing back and forth flow facilitates removal of particulate debris that may be trapped in the flow paths.

FIG. 8E is a diagram of the cleaning system loop 500 with flow in a draining direction 5697. In this step, valve is 5693 is open and the sanitizing solution 5698 is drained out the faucet 404a. In addition, the air pump 5687 operates first, and after a certain pressure is applied inside the sanitizing cap, the valve 5691 is closed and the valve 5693 is opened, so that draining can occur. Note that when valve 5691 is closed, there is a pressure differential on valve 5693 and the used sanitizing solution 5698 can be completely drained. The sanitizing solution is designed so that the system does not need to be flushed with fresh water at the end of the cleaning cycle shown in FIGS. 8A-8E.

FIG. 9A is a cross sectional diagram of a dry hopping module 900 including the separable dry hopping tank 902 according to embodiments of the invention. A system level perspective of an embodiment of the dry hopping module 105 can be visualized with respect to FIGS. 2A and 2B, for example. The dry hopping module 900 may be a single unit or the module may be comprised of separate parts including the separable dry hopping tank 902 and corresponding top lid 905a as shown in FIGS. 9B-9C. The top lid 905a can be positioned on top of the separable dry hopping tank 902 in direction 916 as shown in FIG. 9A.

The separate parts are some components of the dry hopping system 900. The system also includes components such as an airflow line 907, pod structure 908, and multiple gasket seals 911. A reed sensor 914 and level sensor 913 are also included in the dry hopping module 900. A hinge structure 915 allows access to the dry hopping system 900 so that ingredients (e.g., hops, flavoring agents, coloring agents, clarifying agents) can be conveniently added. The separable parts allow for easy cleaning post fermentation.

The dry hopping module 900 is equipped with a check valve 912 at the bottom of the water bottle 917, which can be removed, washed and filled with water. This allows for hygienic control and efficient water supply to the dry hopping module 900. It is also a safer way to clean this area than pouring water directly into the entire fermentation device.

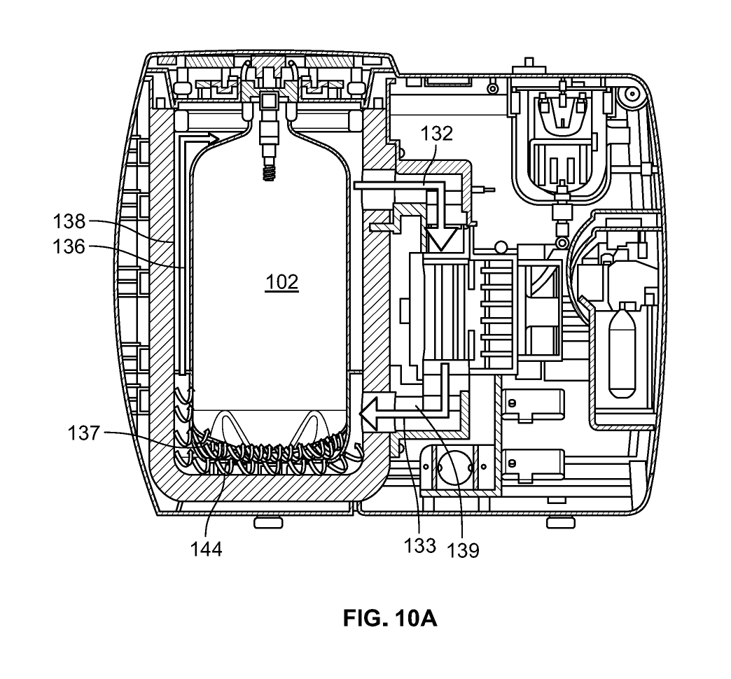

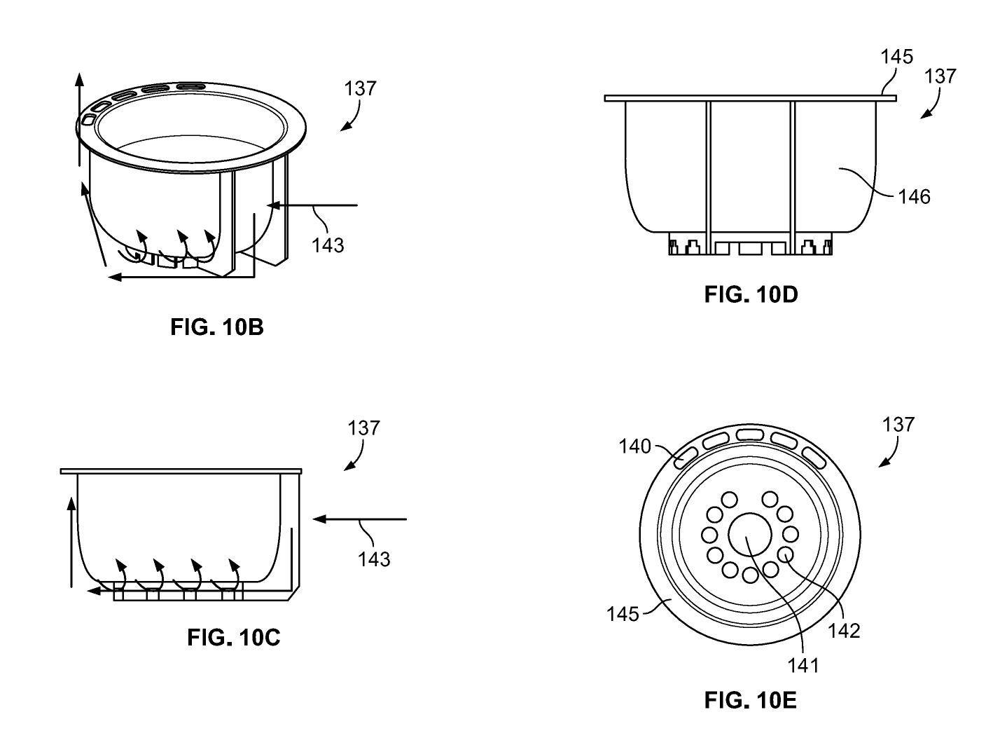

FIG. 10A is a cross sectional diagram showing airflow around the fermentation vessel 102 according to embodiments of the invention. One or more fans 112, as shown in FIG. 2B, may circulate air around the exterior of the fermentation vessel 102 in the general direction 132, 133, 136, when the vessel is positioned in the fermentation device 400, for example.

The fermentation vessel holder (i.e., growler holder) 137 is a structure that distributes airflow more efficiently. The holder 137 also centers and holds the fermentation vessel 102 in line when connecting the locking valve 602 described with respect to FIGS. 6A and 6B. As seen in FIG. 10A, there are airflow guide channels 138, 139 that improve heat exchange. The fermentation holder 137 holds fermentation vessel 102 in the middle of fermentation tank. The fermentation vessel holder 137 has openings 140 along the top edge blade 145 and openings 141, 142 along the body 146 to allow air to circulate freely around the bottom and sides of the fermentation vessel 102 with less resistance. The openings can be any shape or configuration pattern. The air flow direction 143 around the holder 137 can be seen in FIGS. 10B and 10C, for example. Depending on the configuration of openings 140, 141, 142, the air may also flow in vortex, spiral 144, or other non-linear pattern around the bottom and edge of the holder 137 and flow guide channels 138, 139. Many patterns are contemplated each having different heat dissipation outcomes. It is important to directly cool the fermentation vessel and indirectly cool the wort inside because considerable heat may be generated as the wort ferments. The increased air flow efficiently cools the fermentation vessel 102 and the wort.

The center portion of holder 137 around opening 141 and the rest of the body 146 are made of a relatively hard material. The blade 145 is made of a soft elastomer for easy gripping, removal and cleaning as needed.

The special features of the locking cap and check valve assembly described with respect to FIGS. 5A-5B and FIGS. 6A-6B open beer and air flow paths when the cap is secured to the fermentation vessel. If cleaning is desired, the fermentation vessel is detached from the cap and removed from the fermenting device as shown in FIG. 1D. The fermentation vessel can then be washed separately from the device. The rest of the fermenting device can be thoroughly cleaned by first attaching a sanitizing bottle to the cover after the fermentation vessel has been removed. Embodiments of the invention provide for automatically or manually attaching the sanitizing bottle.

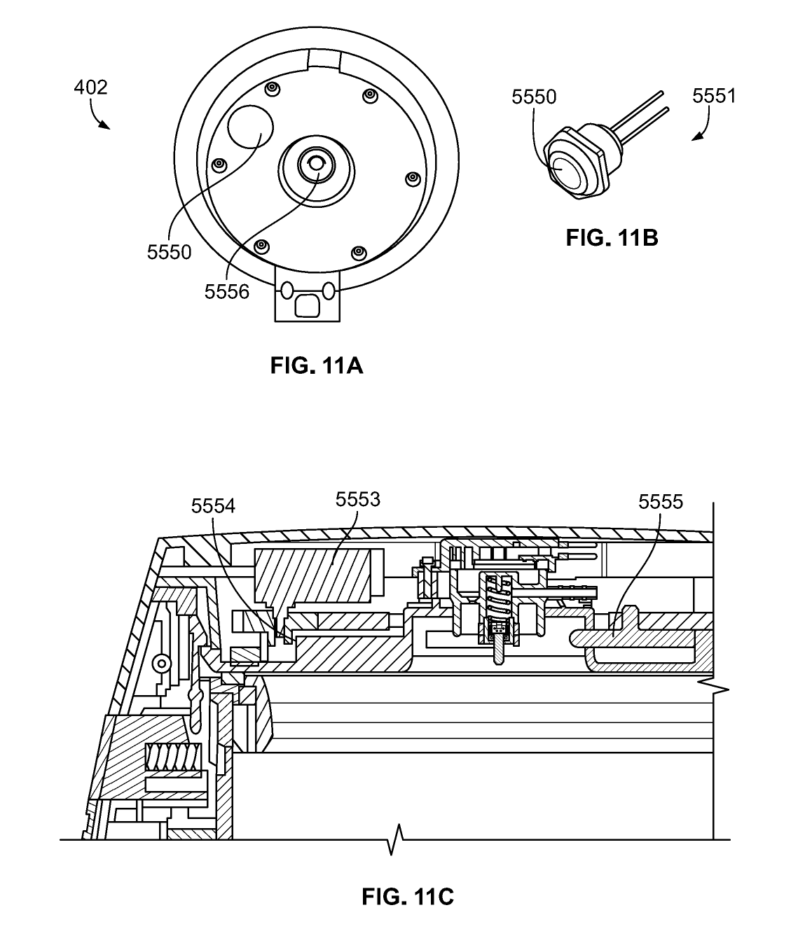

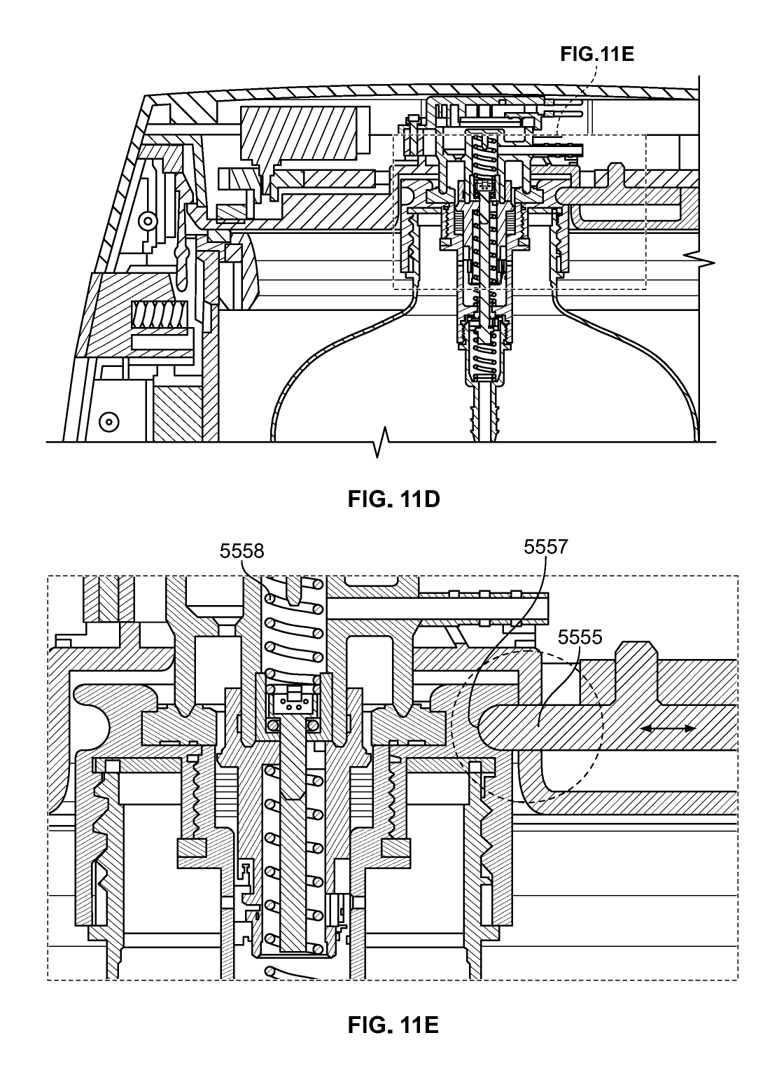

FIG. 11A shows the inside cover 402 of the automatic version of the locking system. A switch 5551 is connected to a locking cap and check valve inside cover 402 of the fermenting device 400. As shown in FIG. 11B, a button 5550 on a switch 5551 activates a motor 5553 (FIG. C) located inside the lid 402 of the fermenting device 400. The motor helps engage a protruding structure 5556 on the underside of the lid 402 with a groove on the cap. A pin 5555 engages a recess 5557 to lock the cap in place and the check valve assembly 5558 opens the flow paths. When the washing procedure is completed, as previously described with respect to FIGS. 8A-8E, the button 5550 is pressed to release the used sanitizing bottle. The fermentation vessel can then be attached and a new round of making fermented beverages can begin.

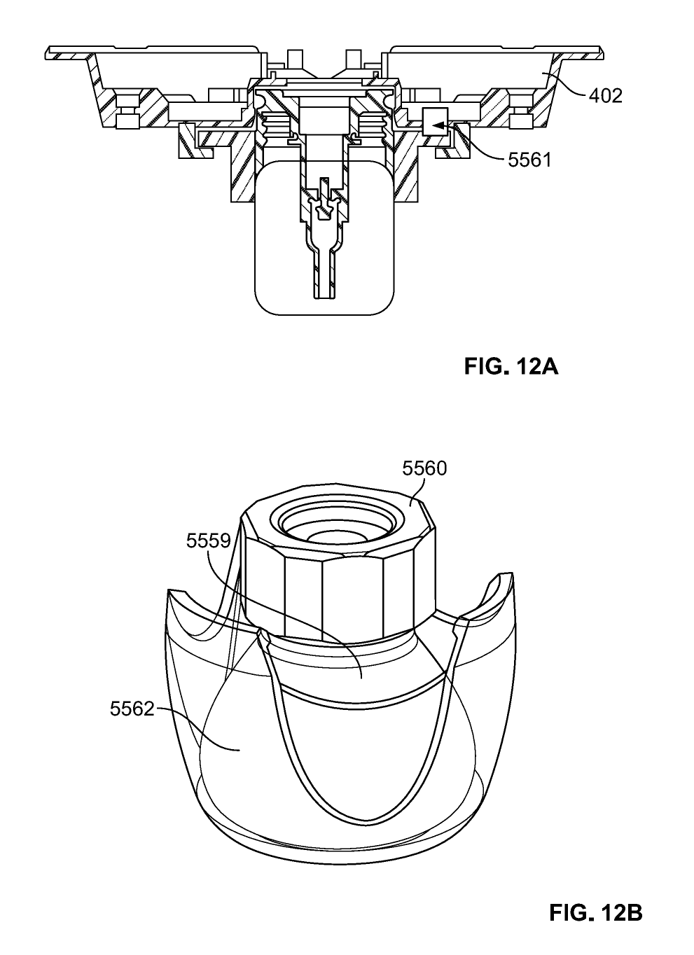

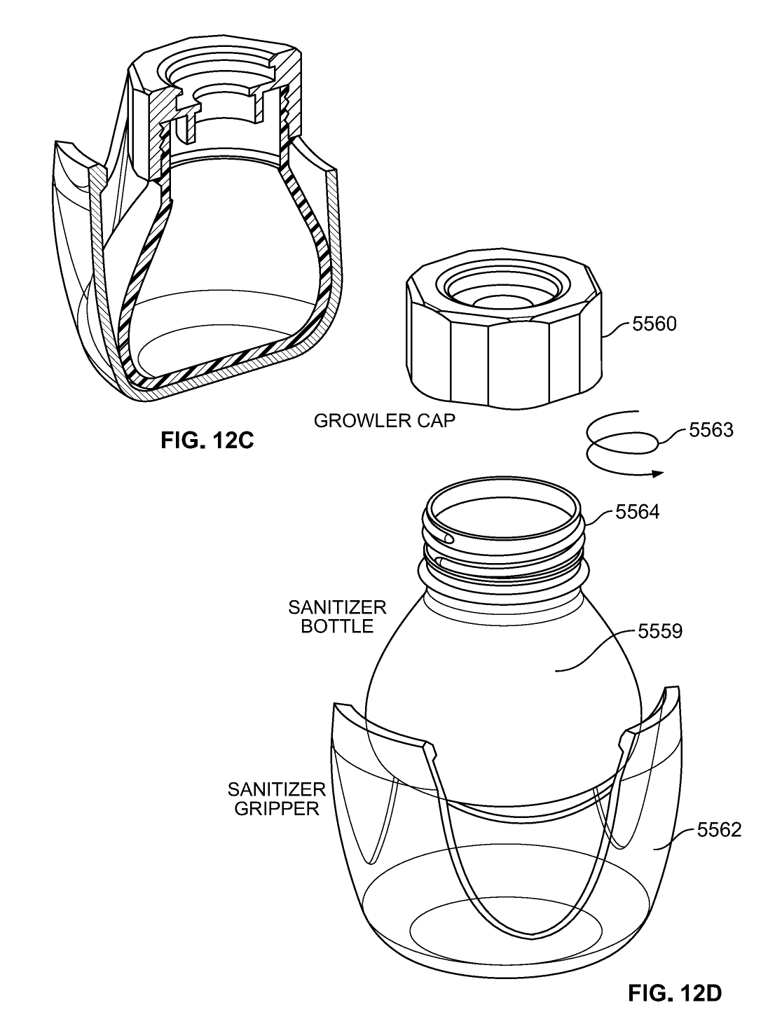

FIG. 12A is a cross sectional diagram of the manual version of the lock system of the portable fermenting device according to embodiments of the invention. A tapered guide, such as a thread, is included on the inside cover 402 of the device. The sanitizing bottle 55559 is configured to engage with the tapered guide. The user is guided by connecting the special cap 5560 and sanitizing bottle 5559 (or fermentation vessel 406 post cleaning) to the hole with the guide in the lid. There may also be an optional switch 5561 near a contact point that confirms a proper connection to the user.

FIG. 12B is a perspective view of the cap 5560, sanitizer bottle 5559, and sanitizer gripper 5562 of the manual lock system and FIG. 12C is a cut-a-way perspective view of FIG. 12B showing the relative positions of each component (i.e., the cap 5560, sanitizer bottle 5559, and sanitizer gripper 5562). As seen in FIG. 12D, the cap 5560 is attached to the sanitizer bottle 5559 via twisting in direction 5563 until the threads 5564 are engaged. Of course, other attachment means may be used. The end of the cap 5560 that is not attached to the sanitizer bottle 5559 is attached manually attached to the lock system shown in FIG. 12A.

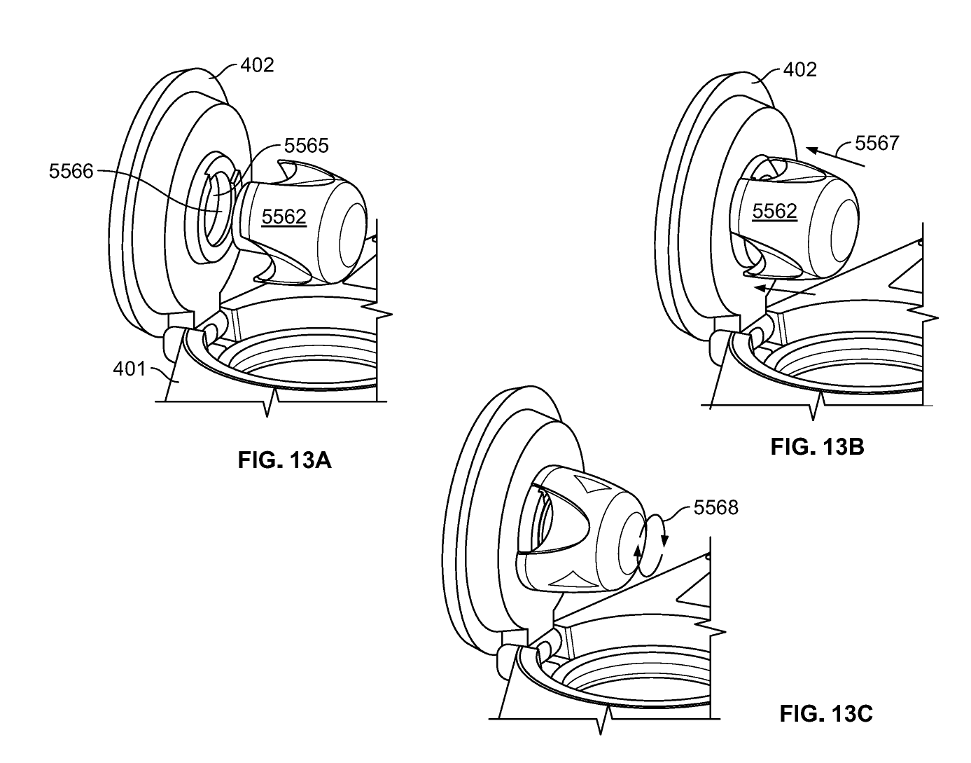

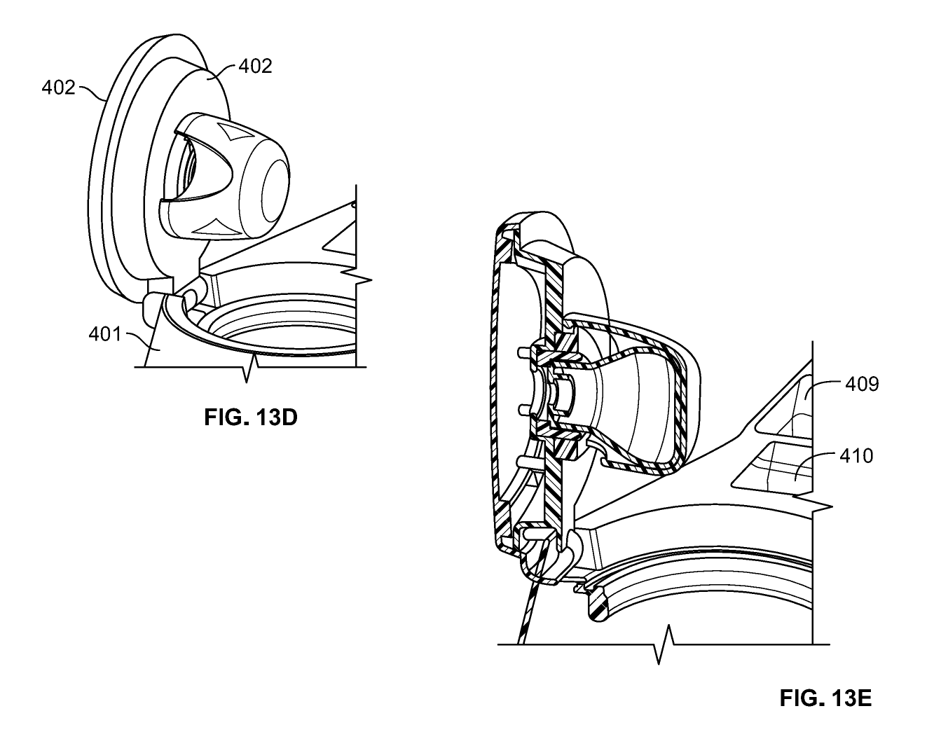

Referring now to FIGS. 13A-13C, the sanitizer gripper 5562 assists with manual installation of the sanitizer bottle 5559. A tapered guide 5565, such as a thread, is included on the inside cover 402 of the device. The sanitizing bottle 5559 is configured to engage with the tapered guide 5565. The user is guided by connecting the special cap 5560 and sanitizing bottle 5559 (or fermentation vessel 406 post cleaning) to the hole 5566 with the guide 5565 in the cover 402. The bottle 5559 is firmly pushed into the locking mechanism in direction 5567 and held in place before twisting the bottle 5556 in direction 5568 the by gripping the sanitizer gripper 5562. This secures the bottle 5559 to the cover 402 as show in FIGS. 13D and 13E, for example. When the valve system is activated, the check valve assembly opens the beer and air flow pathways. This, in turn, allows pathways for the cleaning solution contained in the sanitizing bottle to flow through the device and clean it.

FIG. 14A is a perspective view of the fermentation vessel 102 detached from a mobile dispenser according to embodiments of the invention. The mobile dispenser 5676 allows a fermented beverage to be served when the fermentation vessel 102 is removed from the fermentation device. This adds additional portability for picnics, camping, tailgating and the like. Because the beverage has finished fermentation, the device and electricity to power the device are not needed. Furthermore, when two vessels are used in tandem, one vessel can be configured with the mobile dispenser 5676 while the other vessel is attached to the fermentation system. Alternating the vessels in this manner ensures a steady supply of freshly fermented beverages, including beer, without interruption. The dispenser 5676 includes a tap pull 405b, a faucet 404b, a CO₂ cylinder 5671b, CO₂ regulator 5675, relief valve 5678, a connector 5679, and a locker 5680. An optional CO₂ cylinder cover 5681 is also provided. FIGS. 14A and 14B show one configuration of these components while FIG. 14C shows an alternative configuration.

The mobile dispenser 5676 attaches to the vessel 102 by moving the dispenser in direction 5677 as shown in FIG. 14A. FIG. 14B shows a partially exploded view of the CO₂ cylinder 5671b and the CO₂ cylinder cover 5681 with the vessel attached to the locker 5680. FIG. 14D shows two lockers in a snap-fit configuration of mobile connecting system 5674 that essentially provides an air tight seal. Of course, other locking mechanisms may be employed. While the mobile connecting system 5674 is used to connect the mobile dispenser to a fermentation vessel 102, the other components in region 5673 are similar to the locking structure shown in FIG. 6A and generally serve the same function as previously discussed with regard to FIG. 6A.

The system and various devices may also include one or more software applications, modules, services, or other elements located within at least one working memory device, including an operating system and application programs, such as a client application or browser. It should be appreciated that alternate embodiments may have numerous variations from that described above. For example, customized hardware might also be used and/or particular elements might be implemented in hardware, software (including portable software, such as applets) or both. Further, connection to other computing devices such as network input/output devices may be employed, including servers, mobile smart phones, tablets and PCs.

The specification and drawings are, accordingly, to be regarded in an illustrative rather than a restrictive sense. However, it will be evident that various modifications and changes may be made thereunto without departing from the broader spirit and scope of the disclosure as set forth in the claims.

Other variations are within the spirit of the present disclosure. Thus, while the disclosed techniques are susceptible to various modifications and alternative constructions, certain illustrated embodiments thereof are shown in the drawings and have been described above in detail. It should be understood, however, that there is no intention to limit the disclosure to the specific form or forms disclosed, but on the contrary, the intention is to cover all modifications, alternative constructions and equivalents falling within the spirit and scope of the disclosure, as defined in the appended claims.

The use of the terms “a” and “an” and “the” and similar referents in the context of describing the disclosed embodiments (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. The term “connected” is to be construed as partly or wholly contained within, attached to, or joined together, even if there is something intervening. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate embodiments of the disclosure and does not pose a limitation on the scope of the disclosure unless otherwise claimed. No language in the specification should be construed as indicating any non-claimed element as essential to the practice of the disclosure.

As used in this disclosure, the phrase “alcoholic beverage” may refer to any fermented drink including wine, beer, hard cider, ale, mead, kefir, kvass, or kombucha, for example. The word “fermentation” may refer to either primary or secondary fermentation processes unless otherwise specified. The word “beer” may refer to any beverage that includes a percentage of alcohol per unit volume resulting from partial or complete fermentation of any grain. The word “wine” may refer to any beverage that includes a percentage of alcohol per unit volume resulting from partial or complete fermentation of any fruit. This definition also pertains to so called “alcohol-free” beer and other beverages that have been de-alcoholized post-fermentation.

Any of the methods described herein may be totally or partially performed with a computer system including one or more processors, which can be configured to perform the steps. Thus, embodiments can be directed to computer systems configured to perform the steps of any of the methods described herein, potentially with different components performing a respective steps or a respective group of steps. Although presented as numbered steps, steps of methods herein can be performed at a same time or in a different order. Additionally, portions of these steps may be used with portions of other steps from other methods. Also, all or portions of a step may be optional. Additionally, any of the steps of any of the methods can be performed with modules, circuits, or other means for performing these steps.

The previously described embodiments of the subject invention have many advantages, including effective and efficient ways to produce high-quality fermented beverages, including beer, in convenient small batches using a compact, portable, and automated unit that monitors the fermentation process and provides continual and reliable data that is both accurate and precise without the need for a professional brewer, expensive equipment, extensive labor or inordinate amounts of time.

Although embodiments of the invention have been described in considerable detail with reference to certain preferred versions thereof, other embodiments are possible. Therefore, the spirit and scope of the appended claims should not be limited to the descriptions of the embodiments above.

What is claimed is:

1. A fermentation device comprising:
a fermentation vessel having an open and a closed fermentation configuration;
an specific gravity measurement system comprising a weight sensor and a pressure sensor, the specific gravity measurement system configured to sense the level of fermentation in the open or the closed fermentation configuration; and
a fermentation controller configured to:
access a desired brix profile;
receive the sensed level of fermentation in the open or the closed fermentation configuration from the specific gravity measurement system, and
adjust a fermentation parameter during a primary and/or a secondary fermentation of a mixture contained in the fermentation vessel in real time.

2. The fermentation device of claim 1, wherein the fermentation parameter comprises at least one of a temperature of the fermentation vessel, a pressure of the fermentation vessel, a specific gravity of the mixture, a brix of the fermentation mixture, and the fermentation configuration.

3. The fermentation device of claim 1, wherein the fermentation controller is further configured to:
receive a weight sensed by the weight sensor of the specific gravity measurement system before primary fermentation begins;
receive the specific gravity of the fermentation mixture calculated from a beginning volume of the mixture in the fermentation vessel and a starting specific gravity obtained from a tag;
convert the weight drop sensed from the weight sensor over a period of time to specific gravity during the primary fermentation using a first formula;
determine a first fermentation status of the mixture in the fermentation vessel based on the converted specific gravity, and
adjust the fermentation parameter during the primary fermentation of the mixture in the fermentation vessel based on the first fermentation status and a desired fermentation status.

4. The fermentation device of claim 3, wherein the fermentation controller is further configured to:
receive a pressure of carbon dioxide sensed by the pressure sensor of the specific gravity measurement system when the fermentation vessel is in the closed fermentation configuration at the secondary fermentation;
receive a volume of carbon dioxide from the fermentation device;
convert the pressure and the volume of carbon dioxide to specific gravity using a second formula;
convert the volume of carbon dioxide to an amount of brix using a third formula;
determine a second fermentation status of the mixture in the fermentation vessel based on brix and/or specific gravity; and
adjust the fermentation parameter during the secondary fermentation of the mixture in the fermentation vessel based on the current fermentation status and a desired fermentation status.

5. The fermentation device of claim 4, wherein the first formula is specific gravity=weight/volume; the second formula is weight change=(from 90 n to 95.74 n); PV=nRT→n=PV/RT; and the third formula is specific gravity change=sugar weight change/initial wort volume.

6. The fermentation device of claim 3, wherein the fermentation controller is configured to determine the first or the second fermentation status of the mixture based on a mean value of the specific gravity during a predetermined time interval.

7. The fermentation device of claim 3, wherein the fermentation controller is configured to determine the first or the second fermentation status of the mixture based on a change in the specific gravity during a predetermined interval.

8. The fermentation device of claim 3, wherein the first or the second fermentation status of the mixture is determined in real time.

9. The fermentation device of claim 3, wherein the tag is a QR code, a barcode, an RFID tag, an NFC tag or any other near field data communication mechanism affixed to an outside surface of the fermentation vessel.

10. The fermentation device of claim 1, wherein the desired fermentation status is determined based on the accessed desired brix profile, wherein the accessed desired brix profile comprises a desired brix profile for a particular type of beer.

11. The fermentation device of claim 3, wherein the first or the second fermentation status comprises at least one of an amount of sugar consumption in the fermentation vessel and a fermentation rate of the mixture in the fermentation vessel.

12. The fermentation device of claim 1, wherein the device is a portable and a self-contained fermentation device.

13. A portable beer fermentation device, the device comprising:
a housing having a main chamber for removably receiving a fermentation growler and an ingredients chamber for removably receiving at least one pod, the ingredients chamber in fluid connection with the fermentation growler;
a pressurized fluid source in fluid communication with the ingredients chamber and the fermentation growler, wherein the pressurized fluid comprises filtered air and the fermentation growler is removably received in the main chamber, and wherein the fermentation growler contains a mixture capable of undergoing fermentation; and
a control module configured to:
actuate the pressurized fluid source to transfer contents of the at least one pod into the fermentation growler,
receive an specific gravity measurement of the mixture, and
control at least one of a temperature and a pressure of the fermentation growler based on the specific gravity measurement so as to cause the mixture to undergo a fermentation to yield a beer.

14. The portable beer fermentation device of claim 13, wherein the at least one pod includes a yeast pod, a hop pod, a flavoring pod, a color pod, and/or a pod with any other ingredients.

15. The portable beer fermentation device of claim 13, wherein the mixture capable of undergoing fermentation includes a malt mixture, liquid malt extract, dry malt extract or wort.

16. The portable beer fermentation device of claim 13, further comprising a dispenser in fluid connection with the fermentation growler, wherein the dispenser is configured to dispense the yielded beer to a desired location outside of the housing.

17. The portable beer fermentation device of claim 13, wherein the ingredients chamber is configured to keep the at least one pod immersed in the mixture contained in the fermentation growler for a predetermined time so as to alter the taste, flavor, aroma, color or other characteristics of the beer.

18. The portable beer fermentation device of claim 13, wherein the device is configured to move the mixture between the fermentation growler and the ingredients chamber multiple times such that the flavor and/or the color of the beer is increased each time the mixture is moved.

19. The portable beer fermentation device of claim 13, wherein the fermentation growler is configured to be disposed within the housing or disposed outside the housing, wherein a mobile dispenser allows beer to be served from the fermentation growler when the growler is disposed outside the housing and a dispensing module allows beer to be served from the fermentation growler when the growler is disposed inside the housing and connected to the portable beer fermentation device.

20. The portable beer fermentation device of claim 13, further including a locking cap configured to seal a fermentation growler and a check valve assembly disposed within the locking cap, the check valve assembly configured to open a beer flow path and a gas flow path of the fermentation device when the locking cap engageably seals the fermentation growler and to close the beer flow path and the gas flow path of the fermentation device when the locking cap disengageably unseals the fermentation growler such that inadvertent release of the beer and/or unintentional entry of contaminants are prevented.

21. The portable beer fermentation device of claim 13, wherein the beer includes any combination of a type and a style of the beer as chosen by a consumer.

22. The portable beer fermentation device of claim 21, wherein the type is selected from the group consisting of an ale and a lager; and
wherein the style is selected from the group consisting of bock, doppelbock, pilsner, rauchbier, porter, stout, iambic, amber, blonde, light, white, pale, red, golden, brown, dark, saison, cream, fruit, bitter, honey, IPA, lime, and strong.

23. The portable beer fermentation device of claim 13, further comprising:
one or more processors; and
a non-transitory computer-readable medium containing instructions that, when executed by the one or more processors, cause the one or more processors to perform operations including:
receive an input, wherein the input controls the desired fermentation; identifies a status of the desired fermentation; and notifies a completion of the desired fermentation based on the input.

24. The portable beer fermentation device of claim 23, further comprising instructions that, when executed by the one or more processors, cause the one or more processors to perform operations including:
place orders for supplies;
access information about fermented beverages;
access food pairing information;
purchase accessories;
make recommendations;
connect with social media; and
provide feedback.

25. The portable beer fermentation device of claim 24, wherein the instructions are modified by the one or more processors based on feedback received from at least one sensor, the at least one sensor configured to monitor parameters; and
wherein the at least one sensor is selected from the group consisting of a temperature sensor, a weight sensor, a pressure sensor, an identification sensor, an air quality sensor, an SG sensor, and a level sensor; and wherein the parameters are selected from the group consisting of temperature, weight, pressure, orientation, yeast, gas, and water.

26. A method for making a fermented beverage using a portable fermentation device, the method comprising:
providing a portable fermentation device;
accessing a recipe;
placing primary ingredients into a fermentation vessel, the vessel removably positioned inside the portable fermentation device;
adjusting at least one fermentation parameter;
opening the fermentation vessel;
fermenting one or more of the primary ingredients;
measuring brix and/or specific gravity of the ingredients;
determining fermentation status based on the step of measuring brix and/or specific gravity of the ingredients;
closing the fermentation vessel at a desired fermentation;
optionally adding carbonation;
adjusting a serving temperature; and
dispensing the fermented beverage from the portable fermentation device so as to be enjoyed by a consumer.

27. The method of claim 26, further comprising:
optionally placing additional ingredients into a dry hopping module after the step of determining fermentation status based on brix and/or specific gravity measurement and before the step of closing the fermentation vessel; and
optionally aging after the step of adding carbonation and before the step of adjusting a serving temperature.

28. The method of claim 26, further comprising:
adding secondary ingredients to a dry hopping system of the portable fermentation device before the step of dispensing the fermented beverage.

29. The method of claim 26, wherein the secondary ingredients include hops, flavoring agents, coloring agents, clarifying agent, or any combination thereof.

30. The method of claim 26, wherein the primary ingredients include yeast and at least one substance capable of undergoing fermentation.

31. The method of claim 30, wherein the at least one substance capable of undergoing fermentation is a grain-based wort or a fruit and the fermented beverage is a beer or a wine, respectively.

32. The method of claim 26, wherein the accessing a recipe includes accessing a pre-programed recipe or customizing a recipe to fit a personal taste preference of the consumer.

33. The method of claim 27, further comprising:
replacing the fermentation vessel with a sanitizing cap;
replacing the optional additional ingredients with a sanitizing pod; and
adding water to dry hopping module to begin cleaning the fermentation device.
